(19) United States
(12) Patent Application Publication
(10) Pub. No.: US 2009/0311295 A1
(43) Pub. Date: Dec. 17, 2009

(54) PARTICLES WITH HIGH UNIFORM LOADING OF NANOPARTICLES AND METHODS OF PREPARATION THEREOF

(76) Inventors: Edith Mathiowitz, Brookline, MA (US); Ljiljana Kundakovic, Brookline, MA (US); A. Peter Morello, Haddon Township, NJ (US); Michael W. Harrison, Indianapolis, IN (US); Joshua James Reineke, Shelby Township, MI (US)

Correspondence Address: FISH & RICHARDSON PC, P.O. BOX 1022, MINNEAPOLIS, MN, 55440-1022, US

(21) Appl. No.: 12/300,304

(22) PCT Filed: May 14, 2007
(86) PCT No.: PCT/US07/68881

§ 371 Date: May 14, 2009

Related U.S. Application Data
(60) Provisional application No. 60/747,141, filed on May 12, 2006.

(51) Int. Cl. A61K 8/11; C08L 33/12; A61K 9/14; A61K 47/32; A61P 43/00; A61Q 90/00; B01J 13/04; B01J 13/00

(52) U.S. Cl. 424/401; 524/560; 424/489; 514/772.6; 264/4.7; 264/4.6

(57) ABSTRACT

Methods to produce polymeric microparticles containing nanoparticles such, as pigments, dyes and other chromophores for cosmetic use, plastic surgery therapeutic use, and tattoos have been developed. The microparticles contain within the polymer a very uniform dispersion of dye particles. The methods by which the particles are made ensure a homogeneous mixture and high loading. The microparticles are made using air, one of a number of known methods such as phase inversion, solvent evaporation, and melt processing. The improvement is in the use of a method that makes, a stable dispersion of the nanoparticles in the liquid polymer before formation of the microparticles. This is achieved through selection of appropriate solvent, optionally including surfactant, and then subjecting the dispersion to mechanical processing that stabilizes the dispersion within the polymer solvent, so that the nanoparticles remain suspended for at least thirty minutes, in some cases two hours to 48 hours, sometimes up to three months. The mechanical processing can be sonication and/or production of shear forces, for examples, resulting from use of an open blade or rotor stator mixer or milling with a concentric shaft, at a speed such as between 5000 and 25,000 RPM.

### CROSS REFERENCE TO RELATED APPLICATIONS

This application claims priority to U.S. Ser. No. 60/747,141, filed May 12, 2006.

### FIELD OF THE INVENTION

This invention is generally in the field of methods of manufacture of microparticles encapsulating nanoparticles, for example, pigments, dyes, and other chromophores, or therapeutic, prophylactic or diagnostic agents, having increased loading, uniformity of nanoparticles dispersion, and ease of processing.

### BACKGROUND OF THE INVENTION

Tattooing is done by piercing the skin with needles or similar instruments to introduce an ink that includes small particles of pigment suspended in a liquid carrier. During the healing process, some particles of pigment are transdermally eliminated from the skin surface and others are transported by the lymphatic system. What one sees as the tattoo are the remaining particles of pigment located in the dermis where they are engulfed by phagocytic skin cells (such as fibroblasts and macrophages) or are retained in the extracellular matrix. To create a permanent tattoo one implants pigments that are not dissolved or biodegraded.

U.S. Pat. No. 6,814,760, No. 6,881,249, No. 6,800,122 and published U.S. Patent Application Publication Nos. 20030267964 and 20050178287 to Anderson et al. all entitled “Permanent, Removable Tissue Markings” describe chromophore particles for use in formation of tattoos/tissue markings that can be removed by application of a laser or other energy source. Colored microparticles, each including a chromophore and having a specific property that is designed in advance to enable the microparticles to be altered when exposed to a specific energy (for example, electromagnetic radiation such as near-infrared (near-IR), infrared (IR), near-ultra violet (near-UV), or high intensity visible radiation), are implanted into the tissue to form a detectable tissue marking, wherein the tissue marking is permanent until the specific energy is applied to alter the microparticles to change or remove, or both, the detectable marking. The specific energy can be applied only once to change or remove, or both, the detectable marking. In certain embodiments, the colored microparticles include an indispersible, biologically inert coating, a core enveloped within the coating, wherein the core includes the chromophore which is detectable through the coating and is dispersible in the tissue upon release from the microparticle, and, optionally, an absorption component that absorbs the specific energy and that is located in the coating or the core, or both. When the microparticle ruptures, the chromophore is released and disperses in the tissue, thereby changing or removing, or both, the detectable marking. The coating, the chromophore, or the optional absorption component, or any combination thereof, may absorb specific electromagnetic radiation. The coating can be made of or include a metal oxide, silica, glass, fluorocarbon resin, organic polymer, wax, or a combination thereof.

U.S. Patent Application Publication No. 20050172852 by Anderson et al. published Aug. 11, 2005, describes particles for forming variable appearance tissue marking with frequency up-converting, condition-dependent appearance and/or retro-reflective properties. The tissue markings can be permanent or designed in advance to be removable, degrading over time, either naturally or in a predetermined manner. Condition-dependent appearance tissue markings exhibiting, for example, metachromic properties, can be made to darken reversibly or to change color in response to the aggregation of different dyes. Certain tissue markings can be removed, on demand, by obtaining particles each including a variable appearance material designed to enable the particles to be altered, causing either emission of light or decomposition when exposed to a specific energy (for example, electromagnetic radiation, such as near-infrared (near-IR), infrared (IR), near-ultra violet (near-UV), or high intensity visible radiation). Particles for use in variable appearance tissue markings that exhibit retro-reflective properties can be a spherical, corner cube, cubic crystal or cubic crystal fragment shape. Spherical particles can be formed by, for example, melting selected material, placing the molten material on a disc and rapidly spinning the disc (that is by centrifugal dispersion). The spheres may be sorted by, for example, centrifugation or filtration.

It would be advantageous to provide a method and means for improving the uniformity of the size of the microparticles as well as the uniformity of the nanoparticulate dispersion therein, especially with high loadings, as well as facilitating manufacturing. It would also be advantageous to provide methods for decreasing toxicity of pigments, dyes and chromophores. It would also be advantageous to proved methods to decrease aggregation of dyes, pigments and other chromophores within the microparticles. It would be even more desirable to provide particles having more controlled dissolution and removal properties.

It is therefore an object of the present invention to provide methods and means for improving the uniformity of the particles, especially with high loadings, as well as with increased ease of processing.

It is an additional object of the present invention to provide methods for decreasing toxicity of pigments, dyes and other chromophores. It is another object of the present invention to provide methods for decreasing aggregation of pigments, dyes and other chromophores within the microparticles.

It is still a further object of the present invention to provide particles having more controlled dissolution and removal properties.

### SUMMARY OF THE INVENTION

Methods to produce polymeric microparticles containing nanoparticles such as pigments, dyes and other chromophores for cosmetic use, plastic surgery, therapeutic use, and tattoos have been developed. The microparticles contain within the polymer a very uniform dispersion of dye particles. The polymer should be biocompatible, but can be bioerodible or non-bioerodible. The purpose of the polymer is to make particles larger and prevent both diffusion and aggregation of the nanoparticle. In the case of dyes, the polymer houses the dye, increasing its stability, reducing toxicity (decreased uptake by mitochondria) and sensitization of the skin by the dye (decreased uptake by mitochondria) and sensitization of the skin by the dye and masking its charge.

The methods by which the particles are made ensure a homogeneous mixture and high loading. The microparticles are made using any one of a number of known methods such as phase inversion, solvent evaporation, and melt processing. The improvement is in the use of a method that makes a stable dispersion of the nanoparticles in the liquid polymer before formation of the microparticles. This is achieved through selection of appropriate solvent, optionally including surfactant, and then subjecting the dispersion to mechanical processing that stabilizes the dispersion within the polymer solvent so that the nanoparticles remain suspended for at least thirty minutes, in some cases two hours to 48 hours, sometimes up to three months. The mechanical processing can be sonication and/or production of shear forces, for examples, resulting from use of an open blade or rotor stator mixer or milling with a concentric shaft, at a speed such as between 5000 and 25,000 RPM. In the preferred embodiment for making microparticles using solvent evaporation, the polymer is dissolved in a solvent and mixed with surfactant such as oleic acid, and pigment nanoparticles added, then processed at high shear with large volume of aqueous phase containing a surfactant such as polyvinyl alcohol (“PVA”) to form an emulsion. The oil to water phase ratio is typically 1:20 to ensure small microparticle size in the range 1-3 micron. In the preferred embodiment using phase inversion (“PIN”) to make the microparticles, the polymer is mixed with 0.2 to 1 part surfactant, preferably a hydrophobic surfactant such as oleic acid or cholesterol for hydrophobic dyes, and preferably an amphiphilic surfactant such as TWEEN® for hydrophilic dyes. In another preferred embodiment, the polymer is melted together with dye, without solvent, changing the properties of the dye to make the dye water insoluble, then the dye is dissolved in a solvent such as methylene chloride to make particles using solvent evaporation or PIN. High shear and a long period of vigorous stirring increases drug loading and microparticle uniformity.

### BRIEF DESCRIPTION OF THE DRAWINGS

FIG. 1 shows the stability of α-Fe₂O₃ suspended in PMMA solutions containing different emulsifiers and a solvent:co-solvent ratio (DCM:EA) of 7:3. The most stable iron dispersion (Brij® 92) is ranked “1” while the least stable dispersion (Arlacel® 81) is ranked “8”.

### DETAILED DESCRIPTION OF THE INVENTION

As used herein, “nanoparticle” refers to particle or a structure in the nanometer (nm) range, typically from about 1 to about 1000 nm in diameter, which is encapsulated within the polymer. The terms “nanoparticle” and “material to be encapsulated” are used interchangeably.

As used herein, a “microparticle” is a particle of a relatively small size, but not necessarily in the micron size range; the term is used in reference to particles of sizes that can be, for example, implanted to form tissue markings, and thus can be less than 50 nm to 100 microns or greater. As used herein, the microparticle encompasses microspheres, microcapsules and microparticles, unless specified otherwise. The relative sizes of microparticles and nanoparticles in the context of the present invention are such that the latter can be incorporated into the former. A micro- or nanoparticle may be of composite construction and is not necessarily a pure substance; it may be spherical or any other shape.

As used herein, highly homogeneous microparticles containing nanoparticles in the size range of less than 800 nm are populations wherein 90% of the population having a volumetric average of less than two microns, with a standard deviation of plus or minus 250 nm. Similar percentages and comparatives sizes and variations are applicable to other size ranges of microparticles containing nanoparticles.

The nanoparticle dispersion is stable for a period of time from 0.5 hours to several months, preferably from 0.5 hours to 96 hours, more preferably from 0.5 hours to 48 hours. “Stable”, as used herein, means that the nanoparticles show little or no sedimentation in the polymer solution or molten polymer over the desired period of time.

As used herein, high nanoparticle loadings are loadings of at least from about 10% to about 80%, or from about 10% to about 60%, or at least 40% by weight of the microparticles.

“Color” is broadly defined herein as a detectable (that is, visible or able to be made visible under certain lighting conditions, or able to be detected using a device, for example, an infrared camera) property determined by a substance's electromagnetic absorption and/or emission spectrum (that is, in the ultraviolet, near-ultraviolet, visible, near-infrared, infrared, and other ranges). Black and white are colors under this definition.

### I. Nanoparticles and Microparticles

Polymeric microparticles have uniformly dispersed therein nanoparticles. The nanoparticles have a diameter of less than 1000 nanometers (“nm”); less than 800 nm, more preferably less than 200-500 nm; even more preferably smaller than 100 nm, and most preferably greater than 20 nm. Dye particles in the polymeric microparticles are generally smaller than 100 nm and preferably smaller than 20 nm.

In a preferred embodiment, the microparticles have a diameter of less than 10 microns, preferably less than three microns, and most preferably a diameter of less than 2 microns, with an average of one micron, or less than one micron. Particles smaller than 1 micron are phagocytosed by skin cells, so that the number density in the skin is higher, resulting in a brighter and more stable tattoo with a narrow size distribution in which 90% having Generally the polymer microparticles will contain between about 1% and about 80%, preferably about 10% to about 60%, and more preferably from about 10% to about 40% nanoparticle by weight of microparticle. In some cases, a desired color can be achieved by dye loading from 1-5% by weight.

A. Nanoparticles

The nanoparticles can be dyes, cosmetic, therapeutic, prophylactic, diagnostic, fragrant, nutraceutical, or flavoring agents.

### 1. Dyes

A “chromophore” is broadly defined herein as a substance (solid, liquid, or gas) that has color or imparts a color to the intact microparticles (including when the substance itself lacks color, for example, a clear gas, but scatters electromagnetic waves, for example, light, and thus may appear colored, for example, white, blue, green, or yellow, depending on its scattering properties) under some conditions, for example, all of the time or after exposure to a certain wavelength (such as in a fluorescent substance). For example, a chromophore can be a fluorescent, phosphorescent, wavelength up-converting, or other substance that may normally be substantially invisible, but that emits ultraviolet, visible, or infrared wavelengths during and/or after exposure to wavelengths from a particular region of the electromagnetic spectrum. A chromophore can also be a substance that reversibly or irreversibly changes color spontaneously or in response to any stimulus or photobleaches when exposed to a specific light energy. For example, a chromophore can be a substance that changes appearance or photobleaches upon simultaneous absorption of multiple photons (for example two photon absorption).

As used herein, a substance (such as a chromophore) is “invisible” when essentially no color can be detected (such as in a tissue marking site) apart from the normal coloration of the substance's surroundings (such as skin or other tissue) by the naked eye under normal lighting conditions, for example, diffuse sunlight or standard artificial lighting. A substance is “undetectable” when it is invisible to the naked eye under normal lighting conditions, and also invisible by the naked eye, or a device, under any other lighting conditions (such as fluorescent, UV, or near-infrared).

Dye particle size, as well as dye particle solubility, are ways of controlling removal rate of these particles, since smaller particles are dissolved or digested faster. The dyes can be fluorescent, chemiluminescent, reflective, in the form of amorphous, crystalline, spherical or reflective particles, or may be colorless until activated. The chromophore can be or include rifampin, beta-carotene, tetracycline, indocyanine green, Evan's blue, methylene blue, FD&C Blue No. 1 (Brilliant Blue FCF), FD&C Green No. 3 (Fast Green FCF), FD&C Red No. 3 (Erythrosine), FD&C Red No. 40, FD&C Yellow No. 5 (Tartrazine), FD&C Yellow No. 6 (Sunset Yellow FCF) or other FD&C and D&C dyes and lakes. A lake is a straight color extended on a substratum by adsorption, coprecipitation, or chemical combination that does not include any combination of ingredients made by simple mixing process. The substratum can be alumina, blanc fixe, gloss white, clay, titanium dioxide, zinc oxide, talc, rosin, aluminum benzoate, calcium carbonate, or any combination of two or more of these. The lakes are also salts prepared from one of the straight colors by combining the color with the basic radical sodium, potassium, aluminum, barium, calcium, strontium, or zirconium. In addition, chromophores include natural pigments, metal oxides (such as synthetic iron oxides and titanium dioxide) and carbon. The chromophore can be any colored substance approved by the United States Food and Drug Administration for use in humans. In certain embodiments, the chromophore can be detected by the naked eye under normal lighting conditions or when exposed to UV near-UV, IR, or near-IR radiation.

Other dyes that can be incorporated into polymer include acid fuchsin, alcian blue, alizarin red s, auramine o, azure a and b, Bismarck brown y, brilliant cresyl blue ald, brilliant green, carmine, cibacron blue 3GA, congo red, cresyl violet acetate, crystal violet, eosin b, eosin y, erythrosin b, fast green fcf, giemsa, hematoylin, indigo carmine, Janus green b, Jenner's stain, malachite green oxalate, methyl blue, methylene blue, methyl green, methyl violet 2b, neutral red, Nile blue a, orange II, orange G, orcein, paraosaniline chloride, phloxine b, pyronin b and y, reactive blue 4 and 72, reactive brown 10, reactive green 5 and 19, reactive red 120, reactive yellow 2, 3, 13 and 86, rose bengal, safranin o, Sudan III and IV, Sudan black B and toluidine blue. Examples demonstrate incorporation of water-soluble dyes indigo, indocyanin green, brilliant blue G, and beta-carotene, as well as water-insoluble dye, copper-phthalocyanin.

In one embodiment, the polymer matrix or coating, the chromophore, or an optional absorption component, or any combination thereof, absorb specific wavelengths of electromagnetic radiation. The matrix and/or coating can include a metal oxide, silica, glass, dye or a combination thereof. The matrix and/or coating can be substantially visibly transparent and absorb near-IR radiation, for example, at a wavelength between about 750 nm and about 2000 nm. The absorption component can be or include Schott filter glass, graphite, carbon, or a metal oxide. The coating can itself absorb, or include an absorption component that absorbs, near-IR, IR, near-UV, or high intensity visible radiation.

In another embodiment, the chromophore is detectable through the matrix and/or coating and is altered upon exposure of the microparticle to the specific energy (such as near-infrared or infrared radiation), so that it is no longer colored. For example, the chromophore can be altered by losing its color or by changing from an initial color to a different color upon exposure to the specific energy. The microparticle can further include a bleaching agent that is released upon exposure of the microparticle to the specific energy, thereby bleaching the chromophore (for example, rendering it substantially invisible). The bleaching agent can be a peroxide, hypochlorite, excited oxygen species, or free radical. The chromophore can be pH-sensitive, and the bleaching agent is an acid, a base, or a buffer capable of effecting a pH transition within the core that bleaches the chromophore. The photobleachable chromophore can be Rose Bengal, rhodamine compounds, coumarin compounds, dye-paired ion compounds, cationic dye-borate anion complexes, or bis(diiminosuccino-nitrilo)metal complexes. The chromophore can be photobleachable only upon absorption of multiple photons. The chromophore can also be thermolabile, and exposure of the microparticle to the specific energy heats and alters the chromophore.

A thermolabile chromophore may be any substance that becomes invisible upon heating through absorption of radiation by the chromophore or a component in contact with the chromophore which indirectly heats it. Thermolabile chromophoric mixtures can also be prepared by mixing a specific chromophore with a thermally initiated activator that releases free radicals upon heating. These free radicals then react chemically with the chromophore to render it invisible. The activators are used in the plastics industry for thermal curing of various plastics.

In some cases it is desirable to have in addition to dye particles, uniformly distributed nanoparticles of the “trigger dye” (or egg-tooth) at low loading. The “trigger dye” is used as an optically active component to initiate laser rupture of microparticles. This is particularly useful for removal of yellow/orange and red tattoos that can not be effectively targeted by existing lasers. The “doping” of microparticles by an optically active component ensures that all the colors can be removed by a single laser wavelength. For example, carbon or iron oxide nanoparticles at low loading (<1 or 2% by weight) can be used as “trigger” dyes. Trigger dye does not alter the appearance (color) of microparticles.

Pigment particles can be magnetic and their removal can be achieved by the methods described in Misbah Huzaira and R. Rox Anderson, Magnette Tattoos, Lasers in Surgery and Medicine, 31:121-128 (2002).

Useful chromophore materials as well as materials for discrete absorption component (target ink) are metallic nanoparticles (such as gold, silver and platinum). These metal nanoparticles and derived nanostructures exhibit specific absorption features associated with the plasmon resonance of conduction electrons confined in the nanoparticle. T he absorption frequency (for example absorption maxima or color) and other absorption features (for example the shape and the width of the absorption peaks) of these nanoparticles and other metallic nanostructures (such as nanoshells) is determined by the type of material, size and shape of nanoparticles and nanostructures, size distribution and the environment that surrounds them (Hutter and Fendler, Advanced Materials, 16(19):1685 (1985). For example, gold nanoparticles have strong plasmon resonance absorption at 520 nm and silver nanoparticles have a plasmon resonance at 390 nm. The absorption cross-section of metallic nanoparticles and nanostructures such as nanoshells is million times larger than that of typical molecular chromophores, resulting in efficient light to heat conversion (Brongersma, Nature Materials, 2: 296 (2002)) sufficient to efficiently rupture dermal cells that contain tattoo ink particles.

Metallic and composite metallic nanoparticles and nanostructures that exhibit plasmon resonance can be prepared as nanospheres, nanoshells, rods, rings, disks and cubes. Several different preparation techniques such as colloidal metallic preparation methods, microemulsions, surfactant stabilized micelles, reverse micelles, surfactant vesicles as well as laser ablation methods, vacuum deposition and electron beam lithography have been described (Hutter and Fendler, Advanced Materials, 16(19): 1685 (2004) and references within).

The metallic nanoparticles and nanostructures can be stabilized by covalently bound thiol and disulfide functionalized monolayers. If desired, the surface of metallic nanoparticles can be functionalized to induce a specifically desired immune response or to target specific surface receptors of antigen-presenting tissue cells to induce surface receptor mediated endocytosis, as discussed in International Publication No. WO2006/019823.

Other useful nanomaterials include metallic nanoshells (Halas et al., Journal of Optical Society of America B, 16(10):1824 (1999); Halas et al, Science, 302: 419 (2003). Nanoshells are optically tunable nanoparticles composed of a dielectric (for example, silica) core coated with an ultra-thin metallic (for example, gold) layer. Gold nanoshells (such as those developed by Nanospectra Biosciences, Inc., Houston, Tex.) have physical properties similar to gold colloid, in particular a strong plasmon resonance. The maximum absorption (plasmon resonance) of nanoshells can be varied over a wide spectral range by varying the ratio of inner to outer diameter of the shell, yielding plasmon resonance tunable from 600nm to greater than 1000 nm (Halas, Nano Letters, 3(10): 1411 (2003). For example, gold silica nanoshells with a 100 nm core and 5 nm shell thickness show maximum absorption at ˜1000 nm, while those having the shell thickness of 20 nm have maximum absorption ˜700 nm.

Single and multiple layer nanoshells ranging in size from few nanometers to few hundred nanometers (for example 800 nm: Halas et al., Science, 302: 419 (2003) can be prepared.

Materials with magnetic properties can also be used to construct discrete absorption component. Specifically, black iron oxide nanoparticles such as superparamagnetic iron oxide, (SPIO, particle size greater than 50 nm), ultrasmall superparamagnetic iron oxide (USPIO, particle size smaller than 50 nm) and monodisperse iron oxide nanoparticles (MION, particle size smaller than 20 nm) can be used. Superparamagnetic iron oxide consists of non-stoichiometric microcrystalline magnetite cores, which are coated with dextrans (in ferumoxides) or siloxanes (in ferumoxsils). The most common form of iron oxide used, is magnetite, which is a mixture of Fe₂O₃ and FeO. A mixture using Fe₃O₄ instead of FeO may also be used. These materials are available as tissue specific MRI contrast agents (Feridex®, Endorem™, GastroMARK®, Lumirem®, Sinerem®, Resovist®). USPIO particles are available as MRI contrast agents (Sinerem®, Combidex®, Clariscan™).

Other materials that may be used as discrete absorption particles with IR and near IR absorption include those disclosed in U.S. Pat. No. 6,800,122. In particular, these materials include, but are not limited to, graphite and other forms of carbon and metal oxides, such as iron oxide (red/brown or black), glasses (BG-7 and KG-3 filter glass made by Schott, Inc.), cyanine dyes (including indocyanine green and other colors), phthalocyanine dyes (green-blue), and pyrylium dyes (multiple colors).

Visible-colored materials that can be targeted by visible radiation include, but are limited to, dispersible colorants approved by Food and Drug Administration (FDA) for use in foods, pharmaceutical preparations, medical devices, or cosmetics, such as the non-soluble salts and lakes of FD&C and D&C dyes, as disclosed in U.S. Pat. No. 6,800,122. Additional FDA approved dyes and colored drugs that can be used to form discrete absorption particles are listed in the Code of Federal Regulations (CFR) for Food and Drugs (see Title 21 of CFR chapter 1, parts 1 99).

The percent loading of the chromophores, activators, and/or trigger dyes is from about 0.1% to about 99.9% by weight of the microparticles, preferably from about 1% to about 80%, more preferably from about 10% to about 60%, and most preferably from about 10% to about 40% by weight of the microparticles.

2. Other Agents that can be Incorporated

Although described herein with reference to dyes, it will be understood that any cosmetic, therapeutic, prophylactic, or diagnostic agent, fragrance, nutraceutical, flavoring or other agent that can form nanoparticles, can be incorporated into the microparticles. In one embodiment, the agent is a local anesthetic, which is released following implantation using a needle or compressed air pressure device. In another embodiment, the microparticles can be used to deliver an immunosuppressant or other pharmaceutical for the treatment of a skin disease such as psoriasis.

The percent loading of the other active agents is from about 0.1% to about 99.9% by weight of the micropartidles, preferably from about 1% to about 80%, more preferably from about 10% to about 60%, and most preferably from about 10% to about 40% by weight of the microparticles.

B. Polymers

Bioerodible or non-bioerodible polymers may be used, so long as they are biocompatible. Preferred non-bioerodible polymers are polyacrylates, polymethacrylates, polypropylene, and polyesters. Preferred bio-erodible polymers are polyhydroxyacids such as polylactic acid and copolymers thereof These are approved for implantation into humans.

Suitable polymers have been described in great detail in the prior art. They include, but are not limited to: polyamides, polycarbonates, polyalkylenes, polyalkylene glycols, polyalkylene oxides, polyalkylene terephthalates, polyvinyl alcohols, polyvinyl ethers, polyvinyl esters, polyvinyl halides, polyvinylpyrrolidone, polyglycolides, polysiloxanes, polyurethanes and copolymers thereof, alkyl cellulose, hydroxyalkyl celluloses, cellulose ethers, cellulose esters, nitro celluloses, polymers of acrylic and methacrylic esters, methyl cellulose, ethyl cellulose, hydroxypropyl cellulose, hydroxy-propyl methyl cellulose, hydroxybutyl methyl cellulose, cellulose acetate, cellulose propionate, cellulose acetate butyrate, cellulose acetate phthalate, carboxylethyl cellulose, cellulose triacetate, cellulose sulphate sodium salt, poly(methyl methacrylate), poly(ethylmethacrylate), poly(butylmethacrylate), poly(isobutylmethacrylate), poly(hexylmethacrylate), poly(isodecylmethacrylate), poly(lauryl methacrylate), poly(phenylmethacrylate), poly(methyl acrylate), poly(isopropyl acrylate), poly(isobutylacrylate), poly(octadecyl acrylate), polyethylene, polypropylene poly(ethylene glycol), poly(ethylene oxide), poly(ethylene terephthalate), poly(vinyl alcohols), poly(vinyl acetate), poly vinyl chloride polystyrene and polyvinylpryrrolidone.

Examples of more preferred non-biodegradable polymers include ethylene vinyl acetate, poly(meth)acrylic acid, polyamides, copolymers and mixtures thereof. Examples of more preferred biodegradable polymers include synthetic polymers such as polymers of lactic acid and glycolic acid, polyanhydrides, poly(ortho)esters, polyurethanes, poly(butic acid), poly(valeric acid), poly(caprolactone), poly(hydroxybutyrate), poly(lactide-co-glycolide) and poly(lactide-co-caprolactone), and natural polymers such as alginate and other polysaccharides including dextran and cellulose, collagen, chemical derivatives thereof (substitutions, additions of chemical groups, for example, alkyl, alkylene, hydroxylations, oxidations, and other modifications routinely made by those skilled in the art), albumin and other hydrophilic proteins, zein and other prolamines and hydrophobic proteins, copolymers and mixtures thereof In general, these materials degrade either by enzymatic hydrolysis or exposure to water in vivo, by surface or bulk erosion. These may be used alone, as physical mixtures (blends), or as co-polymers. The concentration of the polymer is from about 0.1% (w/v) to about 10% (w/v).

The bioerodable polymers may be used to provide delayed or extended release of nanoparticles comprising a diagnostic, therapeutic, or prophylactic agent.

Temporary tattoos (time limited tattoos) made with biodegradable polymers and digestible or dissolvable dyes and pigments disappear spontaneously after a predetermined period of time, eliminating the need for laser tattoo removal. The time-limited tattoo particles can be made to last anywhere from three months to two years.

In some cases, polymer and particles can be immuno-modified as disclosed in International Publication No. WO2006/019823 (“Modified Tissue Marking Pigment and Method for Modifying Tissue Marking Pigment” filed Jul. 14, 2005). For example PEGylated polymers can be used to reduce protein adsorption to the surface of the tattoo inks particles and alter the tissue immune response.

C. Solvents and Surfactants

### 1. Solvents

Typical solvents are organic solvents such as methylene chloride, which leave low levels of residue that are generally accepted as safe. Suitable water-insoluble solvents include methylene chloride, chloroform, carbon tetrachloride, dicholorethane, ethyl acetate and cyclohexane. Additional solvents include, but are not limited to, alcohols such as methanol (methyl alcohol), ethanol, (ethyl alcohol), 1-propanol (n-propyl alcohol), 2-propanol (isopropyl alcohol), 1-butanol (n-butyl alcohol), 2-butanol (sec-butyl alcohol), 2-methyl-1-propanol (isobutyl alcohol), 2-methyl-2-propanol (t-butyl alcohol), 1-pentanol (n-pentyl alcohol), 3-methyl-1-butanol (isopentyl alcohol), 2,2-dimethyl-1-propanol (neopentyl alcohol), cyclopentanol (cyclopentyl alcohol), 1-hexanol (n-hexanol), cyclohexanol (cyclohexyl alcohol), 1-heptanol (n-heptyl alcohol), 1-octanol (n-octyl alcohol), 1-nonanol (n-nonyl alcohol), 1-decanol (n-decyl alcohol), 2-propen-1-ol (allyl alcohol), phenylmethanol (benzyl alcohol), diphenylmethanol (diphenylcarbinol), triphenylmethanol (triphenylcarbinol), glycerin, phenol, 2-methoxyethanol, 2-ethoxyethanol, 3-ethoxy-1,2-propanediol, Di(ethylene glycol)methyl ether, 1,2-propanediol, 1,3-propanediol, 1,3-butanediol, 2,3-butanediol, 1,4-butanediol, 1,2-pentanediol, 1,3-pentanediol, 1,4-pentanediol, 1,5-pentanediol, 2,3-pentanediol, 2,4-pentanediol, 2,5-pentanediol, 3,4-pentanediol, 3,5-pentanediol, and combinations thereof. A preferred alcohol is isopropanol.

### 2. Surfactants

Materials that may be used to formulate the coacervate system comprise anionic, cationic, amphoteric, and non-ionic surfactants. Anionic surfactants include di-(2 ethylhexyl)sodium sulfosuccinate; non-ionic surfactants include the fatty acids and the esters thereof; surfactants in the amphoteric group include (1) substances classified as simple, conjugated and derived proteins such as the albumins, gelatins, and glycoproteins, and (2) substances contained within the phospholipid classification, for example lecithin. The amine salts and the quaternary ammonium salts within the cationic group also comprise useful surfactants. Other surfactant compounds useful to form coacervates include polysaccharides and their derivatives, the mucopolysaccharides and the polysorbates and their derivatives. Synthetic polymers that may be used as surfactants include compositions such as polyethylene glycol and polypropylene glycol. Further examples of suitable compounds that may be utilized to prepare coacervate systems include glycoproteins, glycolipids, galactose, gelatins, modified fluid gelatins and galacturonic acid.

Hydrophobic surfactants such as fatty acids and cholesterol are added during processes to improve the resulting distribution of hydrophobic dyes in hydrophobic polymeric microparticles. Examples of fatty acids include butyric acid, valeric acid, caproic acid, enanthic acid, caprylic acid, pelargonic acid, caprylic acid, undecylic acid, lauric acid, tridecylic acid, myristic acid, pentadecylic acid, palmitic acid, heptadecylic acid, stearic acid, nonadecanoic acid, arachic acid, isocrotonic acid, undecylenic acid, oleic acid, elaidic acid, sorbic acid, linoleic acid, linolenic acid and arachidonic acid.

Hydrophilic surfactants such as amphiphilic solvents like TWEEN® 20 and polyvinyl alcohol improve distribution of hydrophilic dye in the polymers. Amphiphilic surfactants are preferred if the dye is hydrophilic and the polymer is hydrophobic.

Surfactant such as a fatty acid or a pharmacologically acceptable salt thereof is typically added in a ratio of from 0.2 to 1 part by weight of the fatty acid or salt thereof to 1 part by weight of the dye.

### II. Methods of Manufacture

It is desirable to have the nanoparticles uniformly distributed within the polymer microparticle. This has numerous advantages, such as more uniform and controlled release in the case of therapeutics, fragrance, or nutraceuticals, higher loading, and in the case of dyes for tattoo inks, to achieve faster removal from the tissue (by dissolution or digestion) following the removal treatment. In the case of tissue markings, during, for example, a laser treatment, polymer microspheres are ruptured or broken into smaller fragments, so that the chromophore are exposed to the extracellular environment. Uniform distribution of the chromophores within the microspheres ensures a high surface area of microparticle fragments. Therefore, the dye is exposed is more easily removed. In some cases, it is also desirable to have uniformly distributed nanoparticles of the discrete absorption component at low loading. The discrete absorption component is used as an optically active material to initiate laser rupture of the microparticle. This is particularly useful for removal of yellow/orange and red tissue markings that cannot be effectively targeted by existing lasers. The doping of microparticles by an optically active component makes it possible to remove tissue markings of all colors by a single laser wavelength. For example, carbon or iron oxide nanoparticles at low loading (less than about 1 or 2% by weight) can be used as the discrete absorption component. The discrete absorption component preferably does not alter the appearance (color) of microspheres. In some cases it may be desirable to have the dye particles uniformly distributed in the outer ring of the microparticle toward the surface of the microparticle to control the pigment removal rate. For example, with biodegradable polymers the dye particles concentrated towards the surface would be exposed and removed faster upon the partial polymer degradation.

The problem with most methods of manufacture of microparticles is that while the nanoparticles are dispersed initially following addition to polymer solution, the nanoparticles rapidly settle towards the bottom. Then when solvent is removed, the nanoparticles are present more preferentially in one part of the polymer than another. It is extremely difficult to keep the nanoparticles dispersed while at the same time removing the polymer solvent to form the microparticles. Therefore, methods have been developed wherein the nanoparticles are dispersed in the polymer solution so that the solution is “stabilized” so that the nanoparticles stay uniformly distributed within the polymer for a period of time sufficient to form the microparticles. This time may be as short as thirty minutes or as long as three months, more typically between two and 48 hours. The stability is a function of the selection of the solvent composition as well as the method of dispersion. In a method theoretically (if not mechanistically) analogous to beating egg whites, the polymer solution is sonicated or otherwise subjected to shear forces, using an open blade mixer or rotor stator at 5000-25,000 RPM, or milled using a concentric shaft, until stable. Alternatively or in addition, the solvent and surfactant, if present, can be used to alter the surface properties of the nanoparticles so that they remain suspended in the polymer solution. The solvent is then removed to form the microparticles having a uniform dispersion of nanoparticles within the polymer.

There are several processes whereby microparticles can be made, including, for example, spray drying, interfacial polymerization, hot melt encapsulation, phase separation encapsulation, spontaneous emulsion, solvent evaporation microencapsulation, solvent removal microencapsulation, coacervation, and low temperature microsphere formation. One preferred method of preparing microparticles of the hydrophobic agent is phase inversion nanoencapsulation (“PIN”). Another preferred method is solvent evaporation microencapsulation (specifically high oil to aqueous phase ratio to achieve small particles with addition of surfactant such as oleic acid to improve dispersion of the pigment in the oil phase). For PIN, the minimum concentration for surfactants is 0.001% w/v (surfactant to non-solvent). In the examples, 0.004% v/v was used. For solvent evaporation, the minimum concentration is 0.1% w/v (polyvinyl alcohol to water). Another preferred method includes addition of the nanoparticles into the polymer liquefied by melting to ensure uniform distribution. The polymer-nanoparticle melt is then processed into microparticles using PIN or solvent evaporation microencapsulation (melt-PIN and melt-SE processes).

The dispersion of the pigment(s), dyes, or active agents within the polymer matrix can be enhanced by varying: (1) the solvent used to solvate the polymer; (2) the ratio of the polymer to the solvent; (3) the particle size of the material to be encapsulated; (4) the percentage of the pigment relative to the polymer (e.g., drug or pigment loading); and/or Cyclone the polymer concentration.

The following are representative methods for forming microparticles.

Spray Drying

In spray drying, the core material to be encapsulated is dispersed or dissolved in a solution. Typically, the solution is aqueous and preferably the solution includes a polymer. The solution or dispersion is pumped through a micronizing nozzle driven by a flow of compressed gas, and the resulting aerosol is suspended in a heated cyclone of air, allowing the solvent to evaporate from the microdroplets. The solidified microparticles pass into a second chamber and are trapped in a collection flask.

Interfacial Polycondensation

Interfacial polycondensation is used to microencapsulate a core material in the following manner. One monomer and the core material are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

Hot Melt Encapsulation

In hot melt microencapsulation, the core material (to be encapsulated) is added to molten polymer. This mixture is suspended as molten droplets in a nonsolvent for the polymer (often oil-based) which has been heated to approximately 10° C. above the melting point of the polymer. The emulsion is maintained through vigorous stirring while the nonsolvent bath is quickly cooled below the glass transition of the polymer, causing the molten droplets to solidify and entrap the core material.

Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in a polymer such as PLA, PLA/PGA copolymer, or PLA/PCL copolymer microcapsules. The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a microcapsule composed of polymer or copolymer shell with a core of liquid material.

Another polymer of interest, poly(methyl methacrylate) (PMMA), is both biocompatible and inert. As detailed below, one can use the solvent evaporation process to encapsulate a metal oxide (a solid) such as iron (III) oxide, another biocompatible material, in conjunction with PMMA to form colored particles that range from light brown or tan to dark brown and black. As a non-limiting example, iron (III) oxide can also be used with other dyes (e.g. beta-carotene) to co-encapsulate multiple compounds within PMMA particles to form other colors, such as yellow or orange-colored particles. The material can be encapsulated using an emulsifier such as Tween 80®, oleic acid, lecithin, Brij® 92, Span® 80, Arlacel® 83, and Span® 85. Alternatively, the material can be encapsulated without the use of an emulsifier.

In one embodiment, microspheres containing α-Fe₂O₃ are formed of PMMA dissolved in an ethyl acetate-DCM cosolvent. Microspheres made from a solvent mixture having greater than 50% ethyl acetate are more likely to be hollow and have larger mean volumetric particle diameters (e.g., greater than 5 microns) that microspheres made from a solvent mixture having greater than 50% DCM. In another embodiment, microspheres containing α-Fe₂O₃ are formed of PMMA dissolved in an ethyl acetate-DCM solvent mixture and containing Tween® 80. Microspheres prepared in this manner were larger, more porous, and contained α-Fe₂O₃ aligned along the circumferential edges of the microspheres.

Solvent evaporation microencapsulation can result in the stabilization of insoluble drug particles or pigments in a polymeric solution for a period of time ranging from 0.5 hours to several months. Stabilizing an insoluble pigment and polymer within the dispersed phase (typically a volatile organic solvent) can be useful for most methods of microencapsulation that are dependent on a dispersed phase, including film casting, solvent evaporation, solvent removal, spray drying, phase inversion, and many others.

The stabilization of insoluble drug particles or pigments within the polymeric solution could be critical during scale-up. By stabilizing suspended drug particles or pigments within the dispersed phase, said particles or pigments can remain homogeneously dispersed throughout the polymeric solution as well as the resulting polymer matrix that forms during the process of microencapsulation. The homogeneous distribution of drug particles or pigments can be achieved in any kind of device, including microparticles, nanoparticles, rods, films, and other device.

Solvent evaporation microencapsulation (SEM) have several advantages. SEM allows for the determination of the best polymer-solvent-insoluble particle mixture that will aid in the formation of a homogeneous suspension that can be used to encapsulate the particles. SEM stabilizes the insoluble particles or pigments within the polymeric solution, which will help during scale-up because one will be able to let suspensions of insoluble particles or pigments sit for long periods of time, making the process less time-dependent and less labor intensive. SEM allows for the encapsulated particles to remain suspended within a polymeric solution for up to 30 days, which may increase the amount of insoluble material entrapped within the polymeric matrix, potentially improving the physical properties of the drug delivery vehicle. SEM allows for the creation of microparticles or nanoparticles that have a more optimized release of the encapsulated material. For example, if the insoluble particle or pigment is localized to the surface of the microparticle or nanoparticle, the system will have a large ‘burst’ effect. In contrast, creating a homogeneous dispersion of the insoluble pigment or particle within the polymeric matrix will help to create a system with release kinetics that begin to approach the classical ‘zero-ordered’ release kinetics that are often perceived as being ideal in the field of drug delivery). Finally, SEM allows for a higher loading of unencapsulated pigment, helping to create microparticles or nanoparticles that function as contrast agents, cellular markers, permanent skin markings, or bioerodible skin markings.

Solvent Removal Microencapsulation

In solvent removal microencapsulation, the polymer is typically dissolved in an oil miscible organic solvent and the material to be encapsulated is added to the polymer solution as a suspension or solution in organic solvent. Surface active agents can be added to improve the dispersion of the material to be encapsulated. An emulsion is formed by adding this suspension or solution to vigorously stirring oil, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid microcapsules containing core material.

Phase Separation Microencapsulation

In phase separation microencapsulation, the material to be encapsulated is dispersed in a polymer solution with stirring. While continually stirring to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

Coacervation

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref. Dowben, R. General Physiology, Harper & Row, New York, 1969, pp. 142-143.). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

Phase Inversion Nanoencapsulation (“PIN”)

A preferred process is PIN. In PIN, a polymer is dissolved in an effective amount of a solvent. The agent to be encapsulated is also dissolved or dispersed in the effective amount of the solvent. The polymer, the agent and the solvent together form a mixture having a continuous phase, wherein the solvent is the continuous phase. The mixture is introduced into an effective amount of a nonsolvent to cause the spontaneous formation of the microencapsulated product, wherein the solvent and the nonsolvent are miscible. PIN has been described by Mathiowitz et al. in U.S. Pat. Nos. 6,131,211 and 6,235,224. A hydrophobic agent is dissolved in an effective amount of a first solvent that is free of polymer. The hydrophobic agent and the solvent form a mixture having a continuous phase. A second solvent and then an aqueous solution are introduced into the mixture. The introduction of the aqueous solution causes precipitation of the hydrophobic agent and produces a composition of micronized hydrophobic agent having an average particle size of 1 micron or less.

An improved process is demonstrated in the examples. The process uses a mixed solvent including at least one water-insoluble solvent and water that contains a surfactant, such as PVA. The drug is either dissolved or dispersed together with a substance that has a high molecular weight (such as a polymer) into an organic solvent composition, optionally containing non-ionic surfactants of various hydrophilic-lipophilic ratios. The composition is then introduced into an aqueous solution that contains a surfactant like PVA. The water-insoluble solvent forms an oil phase (inner phase) and is stirred into the aqueous solution as a water phase (outer phase). The O/W emulsion is combined with fresh water that contains surfactant such as PVA and is stirred to help aid the solvent evaporation. The aqueous solution contains an activator such as polyvinyl alcohol, whereby the oil phase is enclosed as small droplets within the aqueous solution as shells. The proportion of the water-miscible solvent in the oil phase is from 5% to 95%. An important aspect of this improved method is the use of high shear during the initial mixing phase, which is achievable, for example, using sonication for a period of one hour, with stirring, to uniformly mix in high amounts of dye particles in the polymer liquefied by dissolution or by melting.

Melt Solvent Evaporation Method

In the melt-solvent evaporation method, the polymer is heated to a point of sufficient fluidity to allow ease of manipulation (for example, stirring with a spatula). The temperature required to do this is dependent on the intrinsic properties of the polymer. For example, for crystalline polymers, the temperature will be above the melting point of the polymer. After reaching the desired temperature, the dye or agent is added to the molten polymer and physically mixed while maintaining the temperature. The molten polymer and dye or agent are mixed until the mixture reaches the maximum level of homogeneity for that particular system. The mixture is allowed to cool to room temperature and harden. This may result in melting of the dye in the polymer and/or dispersion of the dye in the polymer. This can result in an increase in solubility of the dye when the mixture is dissolved in organic solvent. The process is easy to scale up since it occurs prior to encapsulation. High shear turbines may be used to stir the dispersion, complemented by gradual addition of pigment into the polymer solution until the desired high loading is achieved. Alternatively the density of the polymer solution may be adjusted to prevent pigment settling during stirring.

This method increases microparticle loading as well as uniformity of the resulting microparticles and of the dye within the microparticles. When a dye is formed into microspheres by double-emulsion solvent evaporation, transfer of the dye from the inner phase to the outer water phase can be prevented. This makes it possible to increase the percentage of dye entrapped within the microspheres, resulting in an increased amount of the drug in the microspheres.

The distribution of the dye in particles can also be made more uniform. This can improve the release kinetics of the dye. Generally, the dye is dissolved or dispersed together with a substance that has a high molecular weight in an organic solvent composition; with or without non-ionic surfactants of various hydrophilic-lipophilic ratios. The composition is introduced into an aqueous solution that contains a surfactant like PVA. The water-insoluble solvent forms an oil phase (inner phase) and is stirred into the aqueous solution as a water phase (outer phase). The O/W emulsion is combined with fresh water that contains PVA and is stirred to help aid the solvent evaporation. The aqueous solution contains an activator such as polyvinyl alcohol, whereby the oil phase is enclosed as small droplets within the aqueous solution as shells.

The present invention will be further understood by reference to the following non-limiting examples.

### Examples

### Materials

Medical grade PMMA (M_{w}=35 kDa; residual MMA monomer<0.1%) was ordered from Vista Optics Ltd. (Widnes, UK), PVA (M_{w}=25 kDa; 88% hydrolyzed) was purchased from Polysciences, Inc. (Warrington, Pa., USA).

Cosmetic grade α-Fe₂O₃ (75-125 nm in diameter) was from Meliorum Technologies, Inc. (Rochester, N.Y., USA).

DCM (DCM; Burdick and Jackson, Muskegon, Mich., USA), ethyl acetate (EA; Mallinckrodt, Hazelwood, Mo., USA), and 1-octanol (Sigma-Aldrich, St. Louis, Mo., USA) were analytical grade solvents.

The emulsifiers used included phosphatidylcholine (lecithin; Spectrum Chemicals, Gardena, Calif., USA) and cis-9-octadecenoic acid (oleic acid; Mallinckrodt, Hazelwood, Mo.); polyoxyethylene 2 oleyl ether (Brij 92), polyoxyethylene-sorbitan monooleate (Tween 80), sorbitan monooleate (Span 80), sorbitan sesquioleate (Arlacel 83), and sorbitan trioleate (Span 85), which were obtained from Sigma-Aldrich.

Particles made by Solvent Evaporation Microencansulation:

### Example 1

### Poly(methyl methacrylate) (PMMA) Microparticles (Less Than 2 Microns in Diameter)

Weigh 500 mg of PMMA (25,000 MW) in a 20-ml glass scintillation vial (1); Add 15 ml of dichloromethane (DCM) to PMMA; Vortex (30 seconds) and sonicate (5 minutes) DCM-polymer solution until solution becomes clear. At this point, the polymer should be completely dissolved and there should be no particulate matter. Pour 250 ml of surfactant, 1.0% poly(vinyl alcohol) (PVA) (MW≈25,000 Da; 88% hydrolyzed), into a 1-L Virtis® flask; Pour 100 ml of 0.5% PVA (MW≈25,000 Da; 88% hydrolyzed) into an 800 ml beaker. Place beaker under impeller (approximately 0.5 cm from bottom of beaker) and set impeller speed at 3,000 RPM. Set Virtis® Cyclone to “55” (13,750 RPM); let mix for 60 seconds, then add 100 microliters of 1-octanol. Allow emulsion to set for 5 minutes. Shake polymer solution well and add to Cyclone. Let mix for 15 minutes. Pour contents from Virtis® flask into (6) and let stir for ≈24 hours.

Pour slurry of particles into 50 ml Eppendorf® tubes. Screw cap on and centrifuge for 20 minutes at 4,000 RPM (3345×g); Aspirate off PVA solution using a 50 ml pipette tip; Add 40 ml distilled water to tubes; mix and shake well until particles resuspend in distilled water (NOTE: sonication will likely be necessary to break up particle aggregates stuck to the bottom of the tubes); Screw cap back on and centrifuge for an additional 20 minutes at 4,500 RPM; Aspirate off distilled water using a 50 ml pipette tip; Add 40 ml distilled water to tubes; mix and shake well until particles resuspend in distilled water; Screw cap back on and centrifuge for an additional 20 minutes at 4,000 RPM (3345×g); Aspirate off distilled water using a 50 ml pipette tip; combine slurry of particles into one or two tubes, flash freeze and lyophilize for 48-72 hours.

### Example 2

### Poly(methyl methacrylate) (PMMA) Microparticles Containing 1% Iron (III) Oxide (Less Than 2 Microns in Diameter)

Weigh 500 mg of PMMA (25,000 MW) in a 20-ml glass scintillation vial; Weigh 2.5 mg of iron (III) oxide into a second 20-ml glass scintillation vial; Add 150 microliters of oleic acid to the iron (III) oxide; Add 15 ml of DCM to (2); bath sonicate for 15 minutes; Pour (2) into (1); Vortex (30 seconds) and sonicate (120 minutes) DCM-polymer-iron solution; Pour 250 ml of surfactant, 1.0% PVA (MW≈25,000 Da; 88% hydrolyzed), into a 1-L Virtis® flask; Pour 100 ml of 0.5% PVA (MW≈25,000 Da; 88% hydrolyzed) into an 800 ml beaker. Place beaker under impeller (approximately 0.5 cm from bottom of beaker) and set impeller speed at 3,000 RPM. Set Virtis® Cyclone to “55” (13,750 RPM); let mix for 60 seconds, then add 100 microliters of 1-octanol; allow emulsion to set for 5 minutes; shake polymer solution well and add to Cyclone. Let mix for 15 minutes; pour contents from Virtis® flask into emulsion and let stir for approximately 24 hours.

Collect the particles using the same method illustrated in Example 1.

### Example 3

### Poly(methyl methacrylate) (PMMA) Microparticles Containing 28.5% Iron (III) Oxide and Oleic Acid (Less Than 2 Microns in Diameter)

Weigh 500 mg of PMMA (25,000 MW) in a 20-ml glass scintillation vial; Weigh 200 mg of iron (III) oxide into a second 20-ml glass scintillation vial; Add 250 microliters ofoleic acid to the iron (II) oxide; Add 15 ml of DCM to (2); bath sonicate for 15 minutes; Pour iron oxide into PMMA; Vortex (30 seconds) and sonicate (120 minutes) DCM-polymer-iron solution; Pour 250 ml of surfactant, 1.0% PVA (MW≈25,000 Da; 88% hydrolyzed), into a 1-L Virtis® flask; Pour 100 ml of 0.5% PVA (MW≈25,000 Da; 88% hydrolyzed) into an 800 ml beaker. Place beaker under impeller (approximately 0.5 cm from bottom of beaker) and set impeller speed at 3,000 RPM. Set Virtis® Cyclone to “55” (13,750 RPM); let mix for 60 seconds, then add 100 microliters of 1-octanol; Allow emulsion to set for 5 minutes; Shake polymer solution well and add to PVA. Let mix for 15 minutes; Pour contents from Virtis® flask into emulsion and let stir for approximately 24 hours.

Collect the particles using the same method illustrated in Example 1.

### Example 4

### Poly(methyl methacrylate) (PMMA) Microparticles Containing Iron (III) Oxide (<2 Microns)

Weigh 500 mg of PMMA (25,000 MW) in a 20-ml glass scintillation vial; Weigh 200 mg of iron (III) oxide into a second 20-ml glass scintillation vial; Add 15 ml of DCM to iron oxide; bath sonicate for 15 minutes; Pour DCM into PMMA; Vortex (30 seconds) and sonicate (120 minutes) DCM-polymer-iron solution; Pour 250 ml of surfactant, 1.0% PVA (MW approximately 25,000 Da; 88% hydrolyzed), into a 1-L Virtis® flask; Pour 100 ml of 0.5% PVA (MW≈25,000 Da; 88% hydrolyzed) into an 800 ml beaker. Place beaker under impeller (approximately 0.5 cm from bottom of beaker) and set impeller speed at 3,000 RPM. Set Virtis® Cyclone to “55” (13,750 RPM); let mix for 60 seconds, then add 100 microliters of 1-octanol; Allow emulsion to set for 5 minutes; Shake polymer solution well and add to Cyclone. Let mix for 15 minutes; Pour contents from Virtis® flask into emulsion and let stir for approximately 24 hours.

Collect the particles using the same method illustrated in Example 1.

### Example 5

### Poly(methyl methacrylate) (PMMA) Microparticles Containing 28.5% Iron (II,III) Oxide (Less Than 2 Microns in Diameter)

The protocol is the same as the protocol used in Example 4 except that one should use 200 mg of iron (II,III) oxide.

### Example 6

### Poly(methyl metbacrylate) (PMMA) Microparticles Containing 28.5% Iron (II,III) Oxide with Oleic Acid (Less Than 2 Microns in Diameter)

The protocol is the same as the protocol used in Example 3 except that one should use 200 mg of iron (II,III) oxide.

### Example 7

### Poly(methyl methacrylate) (PMMA) Microparticles Containing 15% Micronized Beta-Carotene (Less Than 2 Microns in Diameter)

This example uses a method described previously (Mathiowitz et al. U.S. Pat. No. 6,824,791) to micronize beta-carotene crystals so they can be more homogeneously distributed throughout the polymer (PMMA) matrix.

### Preparation of Microparticles:

The protocol is the same as the protocol used in Example 4 except that one should use 90 mg of micronized beta-carotene in step (2) and the DCM-beta-carotene solution should only be bath sonicated for 30 minutes.

### Example 8

### Poly(methyl methacrylate) (PMMA) Microparticles Containing 1% Iron (III) Oxide and 15% Micronized Beta-Carotene (Less Than 2 Microns in Diameter)

Similar to Example 7, this example uses a method described previously (Mathiowitz et al. U.S. Pat. No. 6,824,791) to micronize beta-carotene crystals.

Weigh 500 mg of PMMA (25,000 MW) and 90 mg beta-carotene in a 20-ml glass scintillation vial; Weigh 2.5 mg of iron (III) oxide into a second 20-ml glass scintillation vial; Add 150 microliters of oleic acid to the iron (III) oxide; Add 15 ml of DCM to (2); bath sonicate for 15 minutes; Pour iron oxide into PMMA; Vortex (30 seconds) and sonicate (120 minutes) DCM-polymer-iron-beta-carotene solution; Pour 250 ml of surfactant, 1.0% PVA (MW≈25,000 Da; 88% hydrolyzed), into a 1-L Virtis® flask; Pour 100 ml of 0.5% PVA (MW≈25,000 Da; 88% hydrolyzed) into an 800 ml beaker. Place beaker under impeller (approximately 0.5 cm from bottom of beaker) and set impeller speed at 3,000 RPM. Set Virtis® Cyclone to “55” (13,750 RPM); let mix for 60 seconds, then add 100 microliters of 1-octanol; Allow emulsion to set for 5 minutes; Shake polymer solution well and add to Cyclone. Let mix for 15 minutes; Pour contents from Virtis® flask into (8) and let stir for ≈24 hours.

Collect the particles using the same method illustrated in Example 1.

### Example 9

### Poly(methyl methacrylate) (PMMA) Microparticles Containing 1% Iron (III) Oxide (Less Than 2 Microns in Diameter)

Weigh 1000 mg of PMMA (25,000 MW) in a 20-ml glass scintillation vial; weigh 10 mg of iron (III) oxide (nanosize) in a second 20-ml glass scintillation vial; Add 15 ml of DCM to (2) and add 20-60 μl of oleic acid; Vortex (2)+(3) for 30 seconds and sonicate for five minutes; Add (1) to vial containing DCM-iron (III) oxide; Vortex (30 seconds) and sonicate polymer-iron-solvent solution for 60 minutes. At this point, the polymer should be completely dissolved and there should be minimal amounts of visible particulate matter. Pour 150 ml of 0.5% PVA (MW≈25,000 Da; 88% hydrolyzed) into a medium Virtis® flask; Pour 100 ml of 0.5% PVA (MW≈25,000 Da; 88% hydrolyzed) into a 600 ml beaker. Place beaker under impeller (approximately 0.5 cm from bottom of beaker) and set impeller speed at 3,000 RPM. Set Virtis® to “30” setting; let mix for 30-60 seconds; Shake polymer-iron-DCM solution well and add to Cyclone; then immediately turn Virtis® setting to “60.” Let mix for 45 seconds. Pour contents from Virtis® flask into Cyclone and let stir for >12 hours.

Collect the particles using the same method illustrated in Example 1.

### Example 10

### Poly(methyl methacrylate) (PMMA) Microparticles Containing Iron (II,III) Oxide (Variable Concentration) (Less Than 2 Microns in Diameter)

The protocol used in this example is as written in Example 9 except that one should increase the iron (III) oxide to between 50-400 mg in step (2).

### Example 11

### Poly(methyl methacrylate) (PMMA) Microparticles Containing Iron (II,III) Oxide and Oleic Acid (Less Than 2 Microns in Diameter)

The protocol used in this example is as written in Example 9 except that one should use between 50-400 mg of iron (II,III) oxide and 50-250 microliters of oleic acid.

### Example 12

### Poly(methyl methacrylate) (PMMA) Microparticles Containing 15% Stock Beta-Carotene (Less Than 2 Microns in Diameter)

Weigh 175 mg of stock β-carotene. Weigh 1000 mg of PMMA (25,000 MW) in a 20-ml glass scintillation vial; Add 15 ml of DCM to beta caroten and PMMA; Vortex (30 seconds) and sonicate (20 minutes) DCM-polymer-β-carotene solution until solution becomes a clear, bright orange solution at this point, the polymer-β-carotene should be completely dissolved and there should be no particulate matter; stir for >12 hours.

Collect the particles using the same method illustrated in Example 1.

### Example 13

### Poly(methyl methacrylate) (PMMA) Microparticles Containing 15% Micronized Beta-Carotene (Less Than 2 Microns in Diameter)

Weigh out 92.5 mg of micronized beta-carotene in a 20 ml glass scintillation vial; Add 605.5 mg of PMMA (25,000 MW) to beta-carotene. Add 12 ml of DCM. Vortex (30 seconds) and sonicate (20 minutes) DCM-polymer-beta carotene solution until solution becomes a clear, bright orange solution. At this point, the beta-carotene and polymer should be completely in solution and there should be no particulate matter. Pour 200 ml of 0.5% PVA (MW>5,000 Da; 88% hydrolyzed) into a medium Virtis® flask. Pour 100 ml of 0.5% PVA (MW>5,000 Da; 88% hydrolyzed) into a 600 ml beaker. Place beaker under impeller (approximately 0.5 cm from bottom of beaker) and set impeller speed at 3,000 RPM. Add 5 drops octanol to PVA and set Virtis® to “30” setting; let mix for 30-60 seconds; Shake beta carotene well and add to PVA then immediately turn Virtis® setting to “60.” Let mix for 45 seconds. Pour contents from Virtis® flask into Cyclone and let stir for more than 12 hours.

Collect the particles using the same method illustrated in Example 1.

### Example 14

### Poly(methyl methacrylate) (PMMA) Microparticles Containing 1% Iron (III) Oxide and 15% Micronized Beta-Carotene (Less Than 2 Microns in Diameter)

Weigh out 92.5 mg of micronized beta-carotene and 5 mg of iron (III) oxide in a 20 ml glass scintillation vial; Add 605.5 mg of PMMA (25,000 MW);

Add 12 ml of DCM to beta-carotene-iron oxide; Vortex (30 seconds) and sonicate (20 minutes) DCM-polymer-beta carotene solution until solution becomes a clear, bright orange solution. At this point, the beta-carotene and polymer should be completely in solution and there should be no particulate matter; Pour 200 ml of 0.5% PVA (MW>5,000 Da; 88% hydrolyzed) into a medium Virtis® flask. Pour 100 ml of 0.5% PVA (MW>5,000 Da; 88% hydrolyzed) into a 600 ml beaker. Place beaker under impeller (approximately 0.5 cm from bottom of beaker) and set impeller speed at 3,000 RPM. Add 5 drops octanol to PVA and set Virtis® to “30” setting; let mix for 30-60 seconds. Shake beta-carotene well and add to PVA then immediately turn Virtis® setting to “60.” Let mix for 45 seconds. Pour contents from Virtis® flask into Cyclone and let stir for >12 hours. Collect micronized beta-carotene/PMMA microparticles in similar fashion to spheres collected in Example 12.

TABLE I
Summary of Coulter LS230 Particle Size Results for Examples 1-14

| | | | |
|---|---|---|---|
| Example | Mean ± 1 Standard Deviation | V(90%<): | Polymer Type |

Particles <2 microns (mean particle diameter)

| | | | |
|---|---|---|---|
| 1 PMMA | 1.0 μm ± 0.7 μm (n = 3) | 1.7 μm | PMMA |
| 2 PMMA + iron(III) oxide | 1.0 μm ± 0.8 μm (n = 3) | 1.8 μm | PMMA |
| (1%) | | | |
| 3 PMMA + iron(III) oxide | 0.9 μm ± 0.6 μm (n = 2) | 1.8 μm | PMMA |
| (28.5%) + oleic acid | | | |
| 4 PMMA + iron(III) oxide | 1.0 μm ± 0.8 μm (n = 2) | 1.9 μm | PMMA |
| (28.5%) | | | |
| 5 PMMA + iron(II, III) | Determined by TEM | N/A | PMMA |
| oxide (28.5%) | | | |
| 6 PMMA + iron(II, III) | Determined by TEM | N/A | PMMA |
| oxide (28.5%) + oleic acid | | | |
| 7 PMMA + micronized | Not yet determined | N/A | PMMA |
| beta-carotene (15%) | | | |
| 8 PMMA + micronized | Not yet determined | N/A | PMMA |
| beta-carotene (15%) + iron | | | |
| (III) oxide (1%) | | | |

Particles >2 microns (mean particle diameter)

| | | | |
|---|---|---|---|
| 9 PMMA + iron (III) oxide | 1.9 μm ± 0.9 μm (n = 2) | 3.1 μm | PMMA |
| (1%) | | | |
| 10 PMMA + iron (III) oxide | 6.4 μm ± 5.4 μm (n = 4) | 13.9 μm | PMMA |
| (variable concentration) | | | |
| 11 PMMA + iron(II, III) | 3.0 μm ± 4.2 μm (n = 4) | 6.6 μm | PMMA |
| oxide (28.5%) + oleic acid | | | |
| 12 PMMA + stock beta- | 3.8 μm ± 2.0 μm (n = 2) | 6.5 μm | PMMA |
| carotene (15%) | | | |
| 13 PMMA + micronized | 4.4 μm ± 2.2 μm (n = 3) | 7.4 μm | PMMA |
| beta-carotene (15%) | | | |
| 14 PMMA + stock beta- | 1.9 μm ± 0.9 μm (n = 2) | 3.1 μm | PMMA |
| carotene (15%) + iron (III) | | | |
| oxide (1%) | | | |

Particles made by Phase Inversion Nanoencapsulation (PIN)

| | | | |
|---|---|---|---|
| 15 PMMA + TiO₂ (15%) | 1.5 μm ± 4.0 μm (n = 3) | 3.7 μm | PMMA |
| 16 pFA:SA | 1.4 μm ± 0.9 μm (n = 3) | 2.7 μm | pFA:SA |
| 17 pFA:SA + micronized | 1.0 μm ± 0.9 μm (n = 2) | 2.7 μm | pFA:SA |
| dicumarol (30%) | | | |
| 18 pAA | 520 μm ± 200 μm (n = 6) | 1.3 μm | pAA |
| 19 pAA | 440 μm ± 90 μm (n = 6) | 890 nm | pAA |
| 20 pAA + palladium (II) | Determined by TEM | N/A | pAA |
| acetate (30%) | | | |

### Modified Single Emulsion Solvent Evaporation Method

### Example 15

### Poly(methyl methacrylate) EMMA) Microparticles Containing Iron Oxide (Fe₂O₃); Improving the Dispersion of PMMA-Fe₂O₃ Solutions

Iron (III) oxide, a hydrophilic inorganic compound, disperses best in polar organic solvents. However, PMMA, a hydrophobic polymer, is most soluble in a non-polar organic solvent. In order to improve the dispersion of Fe₂O₃ with PMMA, a blend of ethyl acetate (polar; δ=18.6 MPa) and DCM (non-polar; δ=19.8 MPa) was used that could simultaneously disperse Fe₂O₃ while allowing PMMA to remain soluble. 40 μl of different non-ionic surfactants was then added to the best solvent blend and the surfactant selected that appeared to improve and stabilize the dispersion of Fe₂O₃.

Preparation of PMMA-Fe₂O₃ Solutions

22 batches of PMMA-Fe₂O₃ microparticles were fabricated using a modified single emulsion solvent evaporation method; 11 of these used no surfactant in the dispersed phase and 11 used 40 μof Tween® 80 in the dispersed phase. To begin, 800 mg of medical grade PMMA (M_{w}=35 kDa, Vista Optics) and 200 mg of cosmetic grade Fe₂O₃ (Meliorum) was weighed in a 20 ml scintillation vial. Next, mixtures of ethyl acetate (EA) and DCM (DCM) were added, with batch one having a solvent:solvent (EA:DCM) ratio of 10:0 while batch 11 had a ratio of 0:10. For each successive batch, the solvent amounts were varied by 1 ml (e.g. 10:0, 9:1, 8:2 . . . 0:10; see Table II). After adding the solvents, the PMMA-Fe₂O₃ solutions were bath sonicated for 30 minutes using an Aquasonic bath sonicator (T150 model, VWR, West Chester, Pa.) and the solutions vortexed immediately for 30 seconds before introducing them into the primary emulsion.

### Preparation of Primary Emulsion

With four minutes remaining in the bath sonication step, 250 ml of 1.0% PVA (88% hydrolyzed, M_{w}≈25 kDa; Polysciences, Warrington, Pa.) was sheared in a 1-L Virtis® (Virtis, Gardiner, N.Y.) flask at 30,000 RPM using an open blade attached to a Virtis® IQ² mechanical homogenizer. After one minute of shear, 100 μl of 1-octanol was added to minimize the formation of air bubbles. The emulsion was sheared at 30,000 RPM for an additional four minutes (total initial shear time was five minutes). Next, the PMMA-Fe₂O₃ solution was introduced into the primary emulsion using an 18-gauge stainless steel needle attached to a 50 cc syringe at a flow rate of 30 ml/min. The PMMA-Fe₂O₃ emulsion was sheared at 30,000 RPM for an additional 15 minutes (total shearing time was 20 minutes).

Curing and collection of PMMA-Fe₂O₃ Particles

After shearing, the PMMA-Fe₂O₃ emulsion was poured into a 1-L beaker containing 100 ml of a 0.5% PVA solution and the emulsion stirred overnight at 1500 RPM with an impeller attached to an overhead stirrer (Heidolph Instruments, Cinnaminson, N.J.). After curing the particles, the slurry was poured into 50 ml polypropylene Eppendorf® tubes and centrifuged at 4000 RPM (3345×g) for 20 minutes. The particles were washed with double distilled water (ddH₂O), redispersed using a bath sonicator and vortex machine, and this process repeated twice more. This was to remove residual PVA, residual solvents and unencapsulated iron (III) oxide.

Results

The effects of the cosolvent on the size of the PMMA-Fe₂O₃ microparticies are described in Table II.

TABLE II
Summary of particles size of PMMA-Fe₂O₃ microparticles from
Example 15

| | Dispersed | | | Mean | % | % | % | % |
|---|---|---|---|---|---|---|---|---|
| | phase | | | particle | Theoretical | Theoretical | Actual | Loading |
| | DCM:EA/ | Surfactant/ | % | size/ | loading | loading | loading | Efficiency |
| Batch | ml | μl | Yield | μm | Fe₂O₃ | Fe | Fe | Fe |
| 1 | 0:10 | 0 | 85.8 | 9.2 ± 8.9 | 20.10 | 14.06 | 11.10 | 78.95 |
| 2 | 0:10 | 40 | 82.6 | 10.7 ± 10.4 | 20.35 | 14.23 | 13.80 | 96.97 |
| 3 | 1:9 | 0 | 79.9 | 11.3 ± 9.4 | 20.09 | 14.05 | 11.10 | 79.01 |
| 4 | 1:9 | 40 | 88.9 | 12.4 ± 10.0 | 20.01 | 14.00 | 13.60 | 97.18 |
| 5 | 2:8 | 0 | 85.0 | 10.1 ± 10.0 | 19.99 | 13.98 | 9.71 | 69.46 |
| 6 | 2:8 | 40 | 87.9 | 12.6 ± 12.2 | 20.09 | 14.05 | 10.80 | 76.86 |
| 7 | 3:7 | 0 | 87.9 | 13.6 ± 11.5 | 20.07 | 14.04 | 11.30 | 80.49 |
| 8 | 3:7 | 40 | 88.9 | 11.9 ± 13.3 | 20.09 | 14.05 | 11.90 | 84.71 |
| 9 | 4:6 | 0 | 86.9 | 9.3 ± 10.6 | 20.09 | 14.05 | 9.43 | 67.10 |
| 10 | 4:6 | 40 | 83.8 | 9.0 ± 10.4 | 20.09 | 14.05 | 11.30 | 80.42 |
| 11 | 5:5 | 0 | 82.8 | 3.1 ± 3.6 | 20.08 | 14.04 | 12.30 | 87.58 |
| 12 | 5:5 | 40 | 81.0 | 8.2 ± 9.3 | 20.00 | 13.98 | 9.98 | 71.38 |
| 13 | 6:4 | 0 | 76.7 | 2.3 ± 2.5 | 20.07 | 14.03 | 11.10 | 79.09 |
| 14 | 6:4 | 40 | 75.9 | 3.9 ± 5.4 | 20.02 | 14.00 | 12.30 | 87.86 |
| 15 | 7:3 | 0 | 73.4 | 1.8 ± 2.0 | 20.09 | 14.05 | 11.30 | 80.44 |
| 16 | 7:3 | 40 | 73.8 | 3.0 ± 3.4 | 20.14 | 14.09 | 12.40 | 88.02 |
| 17 | 8:2 | 0 | 60.9 | 1.3 ± 1.0 | 20.08 | 14.04 | 10.20 | 72.63 |
| 18 | 8:2 | 40 | 67.0 | 2.1 ± 2.6 | 20.00 | 13.99 | 12.50 | 89.36 |
| 19 | 9:1 | 0 | 68.9 | 1.4 ± 1.1 | 20.06 | 14.03 | 14.90 | 106.18 |
| 20 | 9:1 | 40 | 65.0 | 1.9 ± 2.1 | 20.00 | 13.99 | 14.20 | 101.51 |
| 21 | 10:0 | 0 | 66.4 | 1.4 ± 1.0 | 20.01 | 14.00 | 13.40 | 107.17 |
| 22 | 10:0 | 40 | 64.8 | 2.2 ± 2.4 | 20.09 | 14.05 | 11.70 | 83.26 |

Dispersion as a Function of Emulsifier

α-Fe₂O₃, a hydrophilic inorganic compound, disperses best in polar organic solvents having moderate to strong hydrogen bonding. In contrast, PMMA—a hydrophobic polymer—is most soluble in non-polar organic solvents that have poor hydrogen bonding. DCM is an excellent solvent for PMMA because the two are miscible but PMMA is only partially soluble in EA (≈105 mg/ml).

In an effort to improve the dispersion of α-Fe₂O₃ within solvent:co-solvent solutions of DCM:EA, we tested an intermediate ratio of DCM to EA (7:3) and incorporated various emulsifiers in the dispersed phase to determine if they could improve the dispersion of α-Fe₂O₃ while allowing PMMA to remain soluble. For all emulsifiers, there is an inverse relationship between the HLB and hydrophobicity; a low HLB implies that it is more hydrophobic and vice versa. With the exception of Tween® 80, all of the emulsifiers selected were moderately hydrophobic (Brij® 92) to very hydrophobic (oleic acid).

The ability of two miscible solvents (DCM and EA) and an emulsifier to improve the dispersion of α-Fe₂O₃ was determined by semi-quantitative image analysis.

Microsphere Fabrication

The microspheres were prepared as follows.

A 1% (w/v) solution of polyvinyl alcohol (PVA, M_{w} 25,000 Daltons, 88% hydrolyzed)) was prepared by placing 10 grams of PVA in a 1 liter beaker and 1000 mL of double distilled (dd) water. The mixture was heated to 60° C. stirred gently (e.g., 300 rpm) until the PVA dissolved. The PVA solution was filtered using a Corning® 1-L filtering system (polystyrene; 0.22 micron cellulose acetate filter) connected to a vacuum. 1200 mg of medical grade PMMA (M_{w} 35,000 Daltons) was placed in a 50 mL polypropylene Eppendorf® tube. 225 mg iron (III) oxide was added to the Eppendorf® tube containing the PMMA in step 2. 10 mL of DCM (or a cosolvent, such as DCM-ethyl acetate) was added to the mixture of step 3. 40 microliters of the emulsifier was added to the mixture of step 4. The mixture was bath sonicated for 30 minutes and the centrifuged for 5 minutes at 2,000 rpm. The elution was decanted into a clean, empty 50 mL polypropylene Eppendorf® tube. 250 mL of the solution from step 1 was poured into a 1 liter Virtis® flask. 100 mL of a 0.5% PVA (M_{w}≅25,000 Daltons; 88% hydrolyzed) (the solution from step 1 was used and diluted to prepare a 0.5% solution) was poured into a 1 liter beaker. The beaker was placed under the impeller of an overhead mixer/homogenizer (approximately 0.5 cm from the bottom of the beaker) and the impeller speed was set at 3,000 rpm. The solution from step 7 was inserted into the Virtis® Cyclone. The Cyclone was set to 15,000 rpm and the cycle started. After 1 minute, 100 μL of 1-octanol was added. The PVA was allowed to shear for 5 minutes. The elution from step 6 was shaken and added to the solution of step 8 using a borosilicate glass pipette. The mixture was mixed for 15 minutes. The contents of the Virtis® flask of step 10 were poured into the solution of step 8 and stirred overnight.

The slurry was poured into 50 mL Eppendorf tubes. The tubes were capped and centrifuged for 15 minutes. The 1% PVA solution from step 12 was removed by aspiration or decanting. 40 mL of dd water was added to the Eppendorf tubes. The tubes were capped and shaken well (vortex) until the particles were resuspended in the distilled water (Note: bath sonication may be necessary to break up particle aggregates stuck to the bottom of the tubes). The tubes in step 15 were centrifuged for 20 minutes at 4,0000 rpm and the distillated water was removed by aspiration. Steps were repeated.

The distilled water was removed by aspiration. The particle slurries were combined, flash frozen, and lyophilized for 48-72 hours.

The yield of the microparticles is typically greater than 80%.

High-resolution (10.1 megapixels), 48-bit images were taken of the dispersions and processed using Adobe Photoshop CS2 (Adobe Systems, San Jose, Calif.) and MetaMorph 7.0 (Molecular Devices, Sunnyvale, Calif.). For each image, the optical density as a function of distance was determined using the line scan function (length=1821 pixels) in MetaMorph 7.0 and normalized to unity. To determine the overall color of the dispersions, the non-normalized pixel intensity as a function of distance was measured and recorded by using data from the red channel of the histogram in Adobe Photoshop CS2.

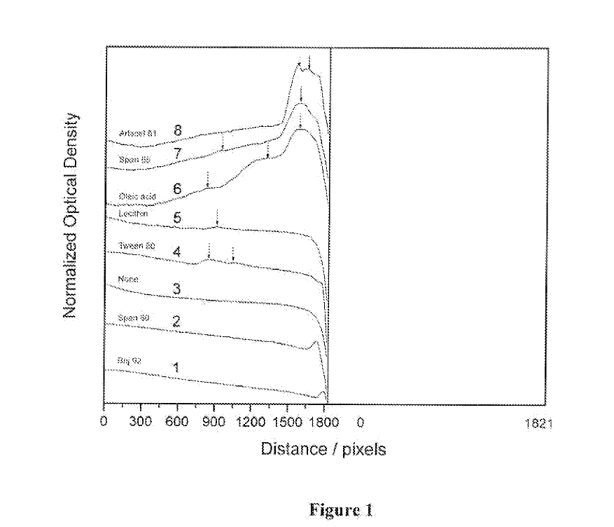

FIG. 1 shows the normalized optical density as a function of distance for eight dispersions (summarized in Table IV). FIG. 1 lists the dispersions with the most sedimentation in descending order: the PMMA-α-Fe₂O₃ dispersion containing Arlacel® 81 was the least stable over two days (ranked eight out of eight) while Brij® 92 produced the most stable PMMA-α-Fe₂O₃ dispersion (ranked one out of eight). The PMMA-α-Fe₂O₃ dispersions made with Span® 85, oleic acid, and Arlacel® 81 were much less stable than those made with lecithin, Tween® 80, no emulsifier, Span® 80, and Brij® 92, producing more iron sedimentation over a two day period. In contrast, the top five most stable dispersions (ranked one through five in FIG. 1) produced little sedimentation over two days. The most stable dispersion appeared to be a homogeneous orange color while the least stable dispersion showed orange color concentrated at the bottom of the vial.

Table IV contains semi-quantitative data measuring the intensity of α-Fe₂O₃ in a solution of PMMA: the most stable iron dispersions had the highest mean intensity and the smallest standard deviation (e.g., Brij® 92, Span® 80, no emulsifier, and Tween® 80) while the least stable dispersions had the smallest intensity and the highest standard deviation.

TABLE IV
Semi-quantitative stability data of α-Fe₂O₃ suspended in the
dispersed phase containing a solvent:co-solvent ratio (DCM:EA) of 7:3. The
units for mean intensity are expressed as the non-normalized optical density
(measured in pixels). Samples that contain more iron dispersed throughout
the solvent mixture have a higher mean intensity and a smaller standard
deviation.

| | | | | | | | Mean | |
|---|---|---|---|---|---|---|---|---|
| | α- | | Solvent | | | | Intensity | |
| PMMA/ | Fe₂O₃/ | Solvent | ratio/ | Emulsifier | | Emulsifier/ | (Red | Dispersion |
| mg | mg | mixture | ml:ml | Type | HLB | μl | channel) | Rank |
| 100 | 50 | DCM:EA | 7:3 | Brij 92 | 4.9 | 100 | 200.8 ± 14.4 | 1 |
| 100 | 50 | DCM:EA | 7:3 | Span 80 | 4.3 | 100 | 204.2 ± 18.4 | 2 |
| 100 | 50 | DCM:EA | 7:3 | None | N/A | 0 | 190.0 ± 18.1 | 3 |
| 100 | 50 | DCM:EA | 7:3 | Tween 80 | 15 | 100 | 188.7 ± 18.5 | 4 |
| 100 | 50 | DCM:EA | 7:3 | Lecithin | 4.6 | 10* | 188.7 ± 18.5 | 5 |
| 100 | 50 | DCM:EA | 7:3 | Oleic acid | 1.0 | 100 | 149.4 ± 41.6 | 6 |
| 100 | 50 | DCM:EA | 7:3 | Span 85 | 1.8 | 100 | 147.6 ± 53.8 | 7 |
| 100 | 50 | DCM:EA | 7:3 | Arlacel 83 | 3.7 | 100 | 123.3 ± 51.6 | 8 |

*Quantity expressed in mg;
N/A = Not applicable

Table V shows the qualitative data of the dispersions after 30 minutes, 2 days, and 30 days at 25° C.±2° C. From Table V, it is evident that dispersions of PMMA-α-Fe₂O₃ that contain Brij® 92 and Span® 80 are the most stable.

TABLE V
Qualitative stability data of α-Fe₂O₃ suspended in the dispersed
phase containing a solvent:co-solvent ratio (DCM:EA) of 7:3 after 30 min, 2
days, and 30 days.

| | | | Solvent | | | Dispersion | Dispersion | Dispersion |
|---|---|---|---|---|---|---|---|---|
| PMMA/ | α-Fe₂O₃/ | Solvent | ratio/ | Emulsifier | Emulsifier/ | Rank | Rank | Rank |
| mg | mg | mixture | ml:ml | Type | μl | (30 min) | (2 days) | (30 days) |
| 100 | 50 | DCM:EA | 7:3 | Brij 92 | 100 | 1 | 1 | 1 |
| 100 | 50 | DCM:EA | 7:3 | Span 80 | 100 | 2 | 2 | 2 |
| 100 | 50 | DCM:EA | 7:3 | None | 0 | 3 | 3 | 3 |
| 100 | 50 | DCM:EA | 7:3 | Tween 80 | 100 | 4 | 4 | 4 |
| 100 | 50 | DCM:EA | 7:3 | Lecithin | 10* | 5 | 5 | 5 |
| 100 | 50 | DCM:EA | 7:3 | Oleic acid | 100 | 6 | 6 | 6 |
| 100 | 50 | DCM:EA | 7:3 | Span 85 | 100 | 7 | 7 | 7 |
| 100 | 50 | DCM:EA | 7:3 | Arlacel 83 | 100 | 8 | 8 | 8 |

Particles made by Phase Inversion Nanoencapsulation (PIN):

Examples 16-18 described below use PIN method described by Mathiowitz et al. in U.S. Pat. Nos. 6,131,211 and 6,235,224. Examples 18-20 described below use an enhanced PIN protocol. The enhanced method utilizes a surfactant in the non-solvent phase to decrease the particle size, increase the homogeneity of the microparticles, increase the loading of dye, and make the dye dispersion more uniform within the microparticle.

### Example 16

### Poly(methyl methacrylate) (PMMA) Microparticles Containing 15% Titanium Dioxide (TiO₂)

Weigh 100 mg of PMMA (25,000 MW) in a 20 ml glass scintillation vial; Add 20 mg of titanium dioxide (TiO₂) to (1); Add 15 ml DCM to (1); Bath sonicate for 30 minutes; Pour polymer-titanium solution into 1000 ml petroleum ether; Filter precipitated product using a cylindrical filter apparatus with a 47 mm 0.22 micron nitrocellulose filter paper; use 15 PSI compressed gas to facilitate faster filtration. Collect particles and lyophilize for 24-48 hours.

### Example 17

### Poly(fumaric-co-sebacic anhydride) (pFA:SA) Particles (<2 Micrometers)

Original PIN Method to fabricate blank (no drug) microparticles using poly(fumaric-co-sebacic) anhydride, or p(FA:SA) and poly(adipic anhydride), p(AA).

Weigh out 100.0 mg of p(FA:SA); Add 10.0 ml DCM Vortex polymer solution (1)+(2) for 60 seconds; Bath sonicate polymer solution (1)+(2) for 60 seconds and let sit for five minutes; Pour 1-L of petroleum ether into a 1-L Pyrex beaker; Pour polymer solution into Cyclone and mix for 30 seconds; Filter precipitated product using a cylindrical filter apparatus with a 47 mm 0.22 micron nitrocellulose filter paper; use 15 PSI compressed gas to facilitate faster filtration.

Collect particles and lyophilize for 24-48 hours.

### Example 18

### Poly(fumaric-co-sebacic anhydride) (pFA:SA) Particles Containing Micronized Dicumarol (Less Than 2 Microns in Diameter)

Original PIN Method to fabricate particles containing drugs using poly(fumaric-co-sebacic) anhydride, or p(FA:SA) and poly(adipic anhydride), p(AA). Weigh out 100.0 mg of p(FA:SA); Weigh out between 0.5 mg and 30 mg of micronized drug and add to polymer. Remainder of protocol the same as Example 16.

### Example 19

### Poly(adipic anhydride) (pAA) Particles (Less Than 1.5 Microns in Diameter)

Prepare Pentane (Petroleum Ether) Bath Containing 0.001% (w/v) Lecithin:

Pour 1000 ml pentane or petroleum ether into 1-L beaker; Add 10 mg of lecithin to (1) and stir for 30 minutes at 500 RPM; keep the beaker covered with aluminum foil so that the solvent does not evaporate (NOTE: pentane and petroleum ether both have a high vapor pressure so they tend to evaporate very quickly—if solvent does evaporate, add more to keep level at 1-L and to keep the concentration of surfactant fixed);

Prepare Polymer-Palladium Solution:

Weigh out 100.0 mg of p(AA) in 20 ml glass scintillation vial; Add 10.0 ml DCM; Vortex polymer solution (1)+(2) for 60 seconds; Bath sonicate polymer solution (1)+(2) for 60 seconds and let sit for five minutes; Draw up polymer solution using a borosilicate glass syringe with luer-lock, injection needle; Keep needle tip 1.5 inches above the pentane (petroleum ether) horizon; Let stream fall into pentane (or petroleum ether) bath; filter precipitated product using a cylindrical filter apparatus with a 47 mm 0.05 or 0.10 micron nitrocellulose filter paper; use 30-40 PSI argon gas to facilitate faster filtration.

### Example 20

### Poly(adipic anhydride) (pAA) Nanoparticles (Less Than 1 Micrometer)

Modified PIN Method to fabricate blank (no drug) particles using p(AA). This protocol is the same as Example 19 except that it uses a 1-L boiling flask instead of a 1-L beaker.

Prepare pentane (petroleum ether) bath containing 0.001% lecithin (w/v). Prepare polymer solution. Weigh out 100.0 mg of p(AA) in 20 ml glass scintillation vial; Add 10.0 ml DCM. Process as described for Example 18.

### Example 21

### Poly(adipic anhydride) (pAA) Nanoparticles Containing 30% Palladium (II) Acetate (Less Than 750 nm Diameter)

Modified PIN Method to fabricate particles using palladium as a model drug using p(AA).

Prepare pentane (petroleum ether) bath containing 0.001% lecithin. Prepare polymer-palladium solution. Weigh out 100.0 mg of p(AA) in 20 ml glass scintillation vial; Weigh out 100.0 mg of palladium (II) acetate and add to polymer; Process as described for Example 20.

Particle sizes, scanning electron micrographs and transmission electron micrographs were obtained for each formulation above. Particle size measurements were taken with a Coulter LS230 particle size machine. A Hitachi 2700 scanning electron microscope (equipped with a lanthanum hexaboride gun) collected micrographs with a Quartz PCI digital imaging system. In addition, transmission electron micrographs were acquired with a Phillips 410 transmission electron microscope equipped with an Advantage HR CCD camera from Advanced Microscopy Techniques (AMT). Images were acquired and analyzed with AMT's imaging software.

### Example 22

### Preparation of Microparticles using a Hot-Melt Process

To perform the melt process, a polymeric material is first heated to a point of sufficient fluidity to allow ease of manipulation with a spatula. The temperature required to do this will vary depending upon the intrinsic properties of the polymeric material. For a crystalline polymer the temperature will be above its melting temperature. Some examples are as follows:

Poly[methyl-methacrylate] (PMMA) of molecular weight (MW) 25 kDa−250 ° C.

Polystyrene (PS) of MW 50 kDa−160 ° C.

Poly[l-lactic]acid (PLLA) of MW 25 kDa−230° C.

After obtaining the relevant temperature, the dye or agent is added to a concentration of between one and 15% w/w and physically mixed with a spatula while maintaining the temperature. The mixing is done until the mixture reaches a maximum level of homogeneity for that system. After this point, the mixture is allowed to cool to 23° C. and hardened.

Brilliant Blue G

Brilliant blue G, also called Coomassie blue, is very water soluble and insoluble in most organics. When melt processed, as described above, it forms a very homogeneous mixture with PS, PMMA and PLLA. The resulting solid mixture is dark navy blue with a smooth, shiny surface. As can be seen from Table III, the dye-melt mixture gains solubility into methylene chloride while maintaining water solubility. Although the process retains the dye's water solubility, the rate of dissolution is slowed.

Encapsulation following the melt process was carried out on brilliant blue G via SE with poly(lactic acid) (“PLLA”) and polymethylmethacrylate (“PMMA”) and via PIN with polystyrene (“PS”) and PMMA. For each of these systems the encapsulation appeared to be successful, yielding baby blue powder formulations that become royal blue following wetting.

Differential scanning calorimetry (DSC) was carried out on the raw materials of PS, PMMA and brilliant blue G. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. Brilliant blue G had no relevant peaks over the scan range. DSC was then performed on the melt mixtures of brilliant blue G with PMMA and PS. None of the polymeric peaks evident in the raw material could be found in the melt mixture for either sample. This indicates that brilliant blue G forms a homogeneous mixture with both PS and PMMA resulting in a completely amorphous sample. DSC on the samples following PIN and SE processing retained this amorphous characteristic. DSC results are summarized in Table III.

X-ray diffraction (XRD) was carried out on the raw materials of PS, PMMA and brilliant blue G. Crystallinity of these materials was calculated by dividing the crystalline area (obtained by subtracting amorphous region) by the total scan area. The resulting crystallinities were 1.1%, 7.1% and 22.3% for PS, PMMA and brilliant blue G, respectively. XRD was then run on the melt mixtures of brilliant blue G with PS and PMMA. The resulting crystallinities were 3.0% and 23.6% for the PS mixture and PMMA mixture, respectively. These crystallinities are consistent with the DSC data in that there is a reduction of crystallinity from what would be expected from a milled mixture. XRD results are summarized in Table III.

Chlorophyll

Chlorophyll is a forest green dye that is water soluble and insoluble in most organics. When melt processed, as described above, it forms a heterogeneous mixture with PS and PMMA. The resulting solid mixture is clear with discrete dark forest green particulates and rough surface. As can be seen from Table III, the dye-melt mixture gains some solubility into methylene chloride, but with a large insoluble fraction, while maintaining water solubility.

No Encapsulation was Attempted for this System.

Differential scanning calorimetry (DSC) was carried out on the raw materials of PS, PMMA) and chlorophyll. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. Chlorophyll had a peak at 14° C. on the cool. DSC was then performed on the melt mixtures of chlorophyll with PMMA and PS. None of the peaks evident in the raw material could be found in the melt mixture for either sample. This indicates that chlorophyll, despite forming a heterogeneous mixture with both PS and PMMA, still results in a completely amorphous sample. DSC results are summarized in Table III.

X-ray diffraction was carried on the raw materials of PS, PMMA and chlorophyll. Crystallinity of these materials was calculated by dividing the crystalline area (obtained by subtracting amorphous region) by the total scan area. The resulting crystallinities were 1.1%, 7.1% and 19.1% for PS, PMMA and chlorophyll, respectively. XRD was then run on the melt mixtures of chlorophyll with PS and PMMA. The resulting crystallinities were 2.6% and 17.3% for the PS mixture and PMMA mixture, respectively. These crystallinities are consistent with the DSC data in that there is a reduction of crystallinity from what would be expected from a milled mixture. XRD results are summarized in Table III.

Copper(II) phthalocyanine

Copper(II) phthalocyanine is a navy blue dye that is water insoluble and insoluble in most organics. When melt processed, as described above, it forms a very homogeneous mixture with PS, PMMA and PLLA. The resulting solid mixture is black with a smooth, shiny surface. As can be seen from Table III, the dye-melt mixture gains solubility into methylene chloride and remains water insoluble.

Encapsulation following the melt process was carried out on copper(II) phthalocyanine via SE and PIN with PS, PLLA and PMMA. For each of these systems the encapsulation appeared to be successful, yielding navy blue powder formulations.

Differential scanning calorimetry (DSC) was carried on the raw materials of PS, PMMA and copper(II) phthalocyanine. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. Copper(II) phthalocyanine had no relevant peaks over the scan range. DSC was then performed on the melt mixtures of copper(II) phthalocyanine with PMMA and PS. It is of interest to note that none of the polymeric peaks evident in the raw material could be found in the melt mixture for either sample. This indicates that copper(II) phthalocyanine forms a homogeneous mixture with both PS and PMMA resulting in a completely amorphous sample. DSC results are summarized in Table III.

X-ray diffraction (XRD) was carried out on the raw materials of PS, PMMA and copper(II) phthalocyanine. Crystallinity of these materials was calculated by dividing the crystalline area (obtained by subtracting amorphous region) by the total scan area. The resulting crystallinities were 1.1%, 7.1% and 67.1% for PS, PMMA and copper(II) phthalocyanine, respectively. XRD was then run on the melt mixtures of copper(II) phthalocyanine with PS and PMMA. The resulting crystallinities were 7.4% and 35.7% for the PS mixture and PMMA mixture, respectively. These crystallinities are consistent with the DSC data in that there is a reduction of crystallinity from what would be expected from a milled mixture. XRD results are summarized in Table III.

Dicumarol

Dicumarol is a hydrophobic anticoagulant that is moderately soluble in most organics. When melt processed, as described above, it forms a very homogeneous mixture with PS and PMMA. The resulting solid mixture is a light milky color with a smooth, shiny surface. The drug-melt mixture retains solubility in methylene chloride.

Differential scanning calorimetry (DSC) was carried on the raw materials of PS, PMMA and dicumarol. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. Dicumarol had a peak at 293° C. in the first heat, 271° C. on the cool and 291° C. on the second heat. DSC was then performed on the melt mixtures of dicumarol with PMMA and PS. It is of interest to note that none of the peaks evident in the raw materials could be found in the melt mixture for either sample. This indicates that dicumarol forms a homogeneous mixture with both PS and PMMA resulting in a completely amorphous sample.

FD&C Blue #1

FD&C blue #1 is a royal blue dye that is very water soluble and insoluble in most organics. When melt processed, as described above, it forms a heterogeneous mixture with PS and PMMA. The resulting solid mixture is clear with discrete dark navy blue particulates and rough surface. As can be seen from Table III, the dye-melt mixture solubility properties do not change.

Differential scanning calorimetry (DSC) was carried out on the raw materials of PS, PMMA and FD&C blue #1. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. FD&C blue #1 had a peak at 107° C. on the first heat. DSC was then performed on the melt mixtures of FD&C blue #1 with PMMA and PS. None of the peaks evident in the raw materials could be found in the melt mixture for either sample. However, in the FD&C blue #1 and PS mixture there was a new peak at 164° C. This indicates that FD&C blue #1, despite forming a heterogeneous mixture with PMMA, still results in a completely amorphous sample. The rise of the new peak in the FD&C blue #1 and PS is of curious note. DSC results are summarized in Table III.

FD&C blue #2

FD&C blue #2 is a royal blue dye that is very water soluble and insoluble in most organics. When melt processed, as described above, it forms a heterogeneous mixture with PS and PMMA. The resulting solid mixture is clear with discrete dark navy blue particulates and rough surface As can be seen from Table III, the dye-melt mixture solubility properties do not change.

Encapsulation following the melt process was carried out on FD&C blue #2 via SE and PIN with PS, PLLA and PMMA. In the SE systems it was immediately obvious that the dye had leached into the aqueous phase and the encapsulation was not successful. In the PIN systems, the encapsulation appeared to be successful, yielding baby blue powder formulations. However, upon wetting the dye quickly went into solution indicating poor encapsulation.

Differential scanning calorimetry (DSC) was carried out on the raw materials of PS, PMMA and FD&C blue #2. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. FD&C blue #2 had no relevant peaks over the scan range. DSC was then performed on the melt mixtures of FD&C blue #2 with PMMA and PS. None of the polymeric peaks evident in the raw material could be found in the melt mixture for either sample. This indicates that FD&C blue #2, despite forming a heterogeneous mixture with both PS and PMA, still results in a completely amorphous sample. DSC results are summarized in Table III.

X-ray diffraction (XRD) was carried out on the raw materials of PS, PMMA and FD&C blue #2. Crystallinity of these materials was calculated by dividing the crystalline area (obtained by subtracting amorphous region) by the total scan area. The resulting crystallinities were 1.1%, 7.1% and 36.4% for PS, PMMA and FD&C blue #2, respectively. XRD was then run on the melt mixtures of FD&C blue #2 with PS and PMMA. The resulting crystallinity for the PS mixture was 11.0%. This crystallinity is consistent with the DSC data in that there is a reduction of crystallinity from what would be expected from a milled mixture. Interestingly, for the PMMA mixture there was no evident spectrum despite multiple runs of the sample. The reason for this is unclear. XRD results are summarized in Table III.

Indocyanine Green

Indocyanine is a forest green dye that is very water soluble and insoluble in most organics. When melt processed, as described above, it forms a very homogeneous mixture with PS, PMMA and PLLA. The resulting solid mixture is dark green to black with a smooth, but sticky texture. As can be seen from the table following the case study section (Table III), the dye-melt mixture gains solubility into methylene chloride while maintaining water solubility.

Encapsulation following the melt process was carried out on indocyanine green via SE and PIN with PS, PLLA and PMMA. For each of these systems the encapsulation appeared to be successful yielding sea-green powder formulations that become forest green following wetting.

Differential scanning calorimetry (DSC) was carried out on the raw materials of PS, PMMA and indocyanine green. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. Indocyanine green had a peak at 254° C. on the first heat and at 47° C. on the cool. DSC was then performed on the melt mixtures of indocyanine green with PMMA and PS. It is of interest to note that none of the polymeric peaks evident in the raw material could be found in the PS melt mixture. This indicates that indocyanine green forms a homogeneous mixture with PS resulting in a completely amorphous sample. However, the PMMA melt mixture has two peaks on the first heat, 152° C. and 168° C., and no other peaks. These peaks do not correspond to any peaks in the raw materials and may indicate a chemical change. This possible chemical change may be due to the fact that the melt process with PMMA takes place at the melting temperature of indocyanine green. DSC on the samples following PIN and SE processing retained an amorphous characteristic with no evident peaks. DSC results are summarized in Table III.

X-ray diffraction (XRD) was carried out on the raw materials of PS, PMMA and indocyanine green. Crystallinity of these materials was calculated by dividing the crystalline area (obtained by subtracting amorphous region) by the total scan area. The resulting crystallinities were 1.1%, 7.1% and 24.8% for PS, PMMA and indocyanine green, respectively. XRD was then run on the melt mixtures of indocyanine green with PS and PMMA. The resulting crystallinity for the PS mixture was 5.2%. This crystallinity is consistent with the DSC data in that there is a reduction of crystallinity from what would be expected from a milled mixture. Interestingly, for the PMMA mixture there was no evident spectrum despite multiple runs of the sample. The reason for this is unclear. XRD results are summarized in Table III.

Iron(III) oxide

Iron(III) oxide is a common ferromagnetic metal compound that is brown or rouge in color. This metal is insoluble in organic and aqueous phases. When melt processed, as described above, it forms a heterogeneous mixture with PS and PMMA. The resulting solid mixture is a light-sand color with a smooth, shiny surface. As can be seen from Table III, the metal-melt mixture maintains its insolubility as expected.

Differential scanning calorimetry (DSC) was carried out on the raw materials of PS, PMMA and iron(III) oxide. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. Iron(III) oxide had no relevant peaks over the scan range. DSC was then performed on the melt mixtures of iron(III)oxide with PMMA and PS. None of the polymeric peaks evident in the raw material could be found in the melt mixture for either sample. This indicates that iron(III) oxide, despite forming a heterogeneous mixture with both PS and PMMA, still results in a completely amorphous sample. DSC results are summarized in Table III.

X-ray diffraction (XRD) was carried out on the raw materials of PS, PMMA and iron(III) oxide. Crystallinity of these materials was calculated by dividing the crystalline area (obtained by subtracting amorphous region) by the total scan area. The resulting crystallinities were 1.1%, 7.1% and 43.2% for PS, PMMA and iron(III) oxide, respectively. XRD was then run on the melt mixtures of iron(III) oxide with PS and PMMA. The resulting crystallinities were 3.6% and 26.9% for the PS mixture and PMMA mixture, respectively. These crystallinities are consistent with the DSC data in that there is a reduction of crystallinity from what would be expected from a milled mixture. XRD results are summarized in Table III.

β-carotene

β-carotene is a red or orange dye is nearly water insoluble and slightly soluble in some organics. When melt processed, as described above, it forms a very homogeneous mixture with PS and PMMA. The resulting solid mixture is bright yellow with a smooth, shiny surface. As can be seen from Table III, the dye-melt mixture gains solubility into methylene chloride and water.

Differential scanning calorimetry (DSC) was carried out on the raw materials of PS, PMMA and β-carotene. These samples were heated, cooled and heated a second time as indicated. PS had a peak at 77° C. in the first heat and 72° C. in the second heat. PMMA had a peak at 120° C. in the first heat, 120° C. on the cool and 124° C. on the second heat. β-carotene had a peak at 40° C. on the cool and 132° C. on the second heat. DSC was then performed on the melt mixtures of β-carotene with PMMA and PS. It is of interest to note that none of the polymeric peaks evident in the raw material could be found in the PS melt mixture. This indicates that β-carotene forms a homogeneous mixture with PS resulting in a completely amorphous sample. However, the PMMA melt mixture has a peak at 61° C. on the cool. This peak does not correspond to any peaks in the raw materials and may indicate a chemical change. This possible chemical change may be due to the fact that the melt process with PMMA takes place above the melting temperature of β-carotene. DSC results are summarized in Table III.

TABLE III
DSC Results

| | | DSC Peaks (° C.) | | | XRD |
|---|---|---|---|---|---|
| | MeCl₂ | | | 2nd | % |
| Raw Material | Solubility | 1st Heat | Cool | Heat | Crystallinity |
| PS | + | 77 | | 72 | 1.1 |
| PMMA | + | 120 | 120 | 124 | 7.1 |
| Brillian blue G | − | | | | 22.3 |
| Chlorophyll | − | | 14 | | 19.1 |
| Copper(II) | − | | | | 67.1 |
| phthalocyanine | | | | | |
| FD&C blue #1 | − | 107 | | | # |
| FD&C blue #2 | − | | | | 36.4 |
| Indocyanine green | − | 254 | 47 | | 24.8 |
| Iron(III) oxide | − | | | | 43.2 |
| beta-Carotene | + | | 40 | 132 | # |
| PS melt w/ | | | | | |
| Brilliant blue G | + | | | | 3.0 |
| Chlorophyll | +/− | | * | | 2.6 |
| Copper(II) | + | | | | 7.4 |
| phthalocyanine | | | | | |
| FD&C blue #1 | − | 164* | | | # |
| FD&C blue #2 | − | | | | 11.0 |
| Indocyanine green | + | * | * | | 5.2 |
| Iron(III) oxide | − | | | | 3.6 |
| beta-Carotene | + | | * | * | # |
| PMMA melt w/ | | | | | |
| Brilliant blue G | + | | | | 23.6 |
| Chlorophyll | +/− | | * | | 17.3 |
| Copper(II) | + | | | | 35.7 |
| phthalocyanine | | | | | |
| FD&C blue #1 | − | * | | | # |
| FD&C blue #2 | − | | | | ! |
| Indocyanine green | + | 152 & | * | | ! |
| | | 168* | | | |
| Iron(III) oxide | − | | | | 26.9 |
| beta-Carotene | + | | 61* | * | # |

Symbol Key
+ denotes soluble
− denotes insoluble
+/− denotes some soluble and insoluble fractions
*denotes change from raw material DSC data
# denotes samples not run on XRD
! denotes samples with no XRD spectrum (samples were run over with same result)

The described melt process has the major advantage of changing the solubility parameters of some agents. However, the solubility seems to be effected only if the agent forms a homogeneous mixture with the polymer. The one exception was chlorophyll, which resulted in a heterogeneous mixture, but its change in solubility was of a small magnitude. DSC data indicated that there may have been a chemical change or reaction that took place when the process was carried out at or above the agent's melting temperature. While all the samples that this occurred in were successfully (changed solubility properties) it does not seem to be a requirement as many other successful melts were made below the agents melting temperature. In all samples, both hetero- and homo-geneous mixtures, there was a reduction in the sample crystallinity and the possible formation of a solid solution.

The following examples are specific protocols that have been used with the melt process.

### Example 23

### Polystyrene (PS) Melt Containing 5-30% Dye

Weigh 870 mg of PS (50,000 MW) in a ceramic crucible; Heat on a hotplate to a temperature of 160° C. and allow PS to melt; Weigh 130 mg (for 13%, but can range from 5-30%) of dye (can be performed with brilliant blue G, carbon, β-carotene, chlorophyll, copper(II) phthalocyanine, FD&C blue #1, FD&C Red #27, indocyanine green, iron(III) oxide, and others); Add dye and mix while on hotplate until a homogeneous mixture is formed; Remove from heat and allow to cool at room temperature; Collect sample in a 20-ml glass scintillation vial.

### Example 24

### Poly(methyl methacrylate) (PMMA) Melt Containing 5-30% Dye

Weigh 870 mg of PMMA (25,000 MW) in a ceramic crucible; Heat on a hotplate to a temperature of 250° C. and allow PMMA to melt; Weigh 130 mg (for 13%, but can range from 5-30%) of dye (can be performed with brilliant blue G, carbon, β-carotene, chlorophyll, copper(II) phthalocyanine, FD&C blue #1, FD&C Red #27, indocyanine green, iron(III) oxide, and others); Add dye to PMMA and mix while on hotplate until a homogeneous mixture is formed; Remove from heat and allow to cool at room temperature.

Collect sample in a 20-ml glass scintillation vial.

### Example 25

### Poly(l-lactic acid) (PLA) Melt Containing 5-30% Dye

Weigh 870 mg of PLA (2,000 MW) in a ceramic crucible; Heat on a hotplate to a temperature of 175° C. and allow PLA to melt; Weigh 130 mg (for 13%, but can range from 5-30%) of dye (can be performed with brilliant blue G, carbon, β-carotene, chlorophyll, copper(II) phthalocyanine, FD&C blue #1, FD&C Red #27, indocyanine green, iron(III) oxide, and others); Add dye to PLA and mix while on hotplate until a homogeneous mixture is formed; Remove from heat and allow to cool at room temperature. Collect sample in a 20-ml glass scintillation vial.

Melt—Phase Inversion Nanoencansulation (mPIN) Method

### Example 26

### Polystyrene (PS) Microparticles Containing 13% Brilliant Blue G (<2 Microns)

Obtain sample of PS—brilliant blue G melt (see melt method protocol above); Add 20 ml of DCM to (I); vortex for 2 minutes; Pour 1 L of petroleum ether into a 1 L glass beaker; Add (2) to (3) and allow suspension to sit for 5 minutes.

Place a Buchner funnel over a vacuum trap; Insert qualitative 5 filter paper (˜2.5 microns) into the Buchner funnel; Pour slurry of particles over filter paper and allow complete drainage of media. Collect microsphere powder from filter paper. Flash freeze and lyophilize for 48 hours.

### Example 27

### Polystyrene (PS) Microparticles Containing 13% β-carotene (<2 Microns)

Obtain sample of PS—β-carotene melt (see melt method protocol above); Add 20 ml of DCM to (1); vortex for 2 minutes; Pour 1 L of petroleum ether into a 1 L glass beaker; Add (2) to (3) and allow suspension to sit for 5 minutes.

Collect the particles using the same method illustrated in Example 1.

### Example 28

### Polystyrene (PS) Microparticles Containing 13% Chlorophyll (<2 Microns)

Obtain sample of PS—chlorophyll melt (see melt method protocol above); Add 20 ml of DCM to (1); vortex for 2 minutes; Pour 1 L of petroleum ether into a 1 L glass beaker; Add DCM to petroleum ether and allow suspension to sit for 5 minutes;

Collect the particles using the same method illustrated in Example 1.

### Example 29

### Polystyrene (PS) Microparticles Containing 13% Copper (II) Phthalocyanine (<2 Microns)

Obtain sample of PS—copper(II) phthalocyanine melt (see melt method protocol above); Add 20 ml of DCM to (1); vortex for 2 minutes; Pour 1 L of petroleum ether into a 1 L glass beaker; Add DCM of petroleum ether and allow suspension to sit for 5 minutes;

Collect the particles using the same method illustrated in Example 1.

### Example 30

### Polystyrene (PS) Microparticles Containing 13% FD&C Blue #1 (<2 Microns)

Obtain sample of PS—FD&C blue #1 melt (see melt method protocol above); Add 20 ml of DCM to (1); vortex for 2 minutes; Pour 1 L of petroleum ether into a 1 L glass beaker; Add (2) to (3) and allow suspension to sit for 5 minutes.

Collect the particles using the same method illustrated in Example 1.

### Example 31

### Polystyrene (PS) Microparticles Containing 13% Indocyanine Green (<2 Microns)

Obtain sample of PS—indocyanine green melt (see melt method protocol above); Add 20 ml of DCM to (1); vortex for 2 minutes; Pour 1 L of petroleum ether into a 1 L glass beaker; Add DCM to petroleum ether and allow suspension to sit for 5 minutes;

Collect the particles using the same method illustrated in Example 1.

### Example 32

### Polystyrene (PS) Microparticles Containing 13% Iron (III) Oxide (<2 Microns)

Obtain sample of PS—iron(III) oxide melt (see melt method protocol above); Add 20 ml of DCM to (1); vortex for 2 minutes; Pour 1 L of petroleum ether into a 1 L glass beaker; Add DCM to petroleum ether and allow suspension to sit for 5 minutes. Collect the particles using the same method illustrated in Example 1.

### Example 33

### Poly(methyl methacrylate) (PMMA) Microparticles Containing 13% Brilliant Blue G (<2 Microns)

Obtain sample of PMMA—brilliant blue G melt (see melt method protocol above); Add 20 ml of DCM to (1); vortex for 2 minutes; Pour 1 L of petroleum ether into a 1 L glass beaker; Add DCM to petroleum ether and allow suspension to sit for 5 minutes. Collect the particles using the same method illustrated in Example 1.

### Example 34

### Poly(methyl methacrylate) (PMMA) Microparticles Containing 13% β-Carotene (<2 Microns)

Obtain sample of PMMA—β-carotene melt (see melt method protocol above); Add 20 ml of DCM to (1); vortex for 2 minutes; Pour 1 L of petroleum ether into a 1 L glass beaker; Add DCM to petroleum ether and allow suspension to sit for 5 minutes.

Collect the particles using the same method illustrated in Example 1.

### Example 35

### Poly(methyl methacrylate) (PMMA) Microparticles Containing 13% chlorophyll (<2 Microns)

Obtain sample of PMMA—chlorophyll melt (see melt method protocol above); Add 20 ml of DCM to (1); vortex for 2 minutes; Pour 1 L of petroleum ether into a 1 L glass beaker; Add (2) to (3) and allow suspension to sit for 5 minutes. Collect the particles using the same method illustrated in Example 1.

### Example 36

### Poly(methyl methacrylate) (PMMA) Microparticles Containing 13% Copper (II) Phthalocyanine (<2 Microns)

Obtain sample of PMMA—copper(II) phthalocyanine melt (see melt method protocol above); Add 20 ml of DCM to (1); vortex for 2 minutes; Pour 1 L of petroleum ether into a 1 L glass beaker; Add (2) to (3) and allow suspension to sit for 5 minutes. Collect the particles using the same method illustrated in Example 1.

### Example 37

### Poly(methyl methacrylate) (PMMA) Microparticles Containing 13% FD&C Blue #1 (<2 Microns)

Obtain sample of PMMA—FD&C blue #1 melt (see melt method protocol above); Add 20 ml of DCM to (1); vortex for 2 minutes; Pour 1 L of petroleum ether into a 1 L glass beaker; Add DCM to petroleum ether and allow suspension to sit for 5 minutes. Collect the particles using the same method illustrated in Example 1.

### Example 38

### Poly(methyl methacrylate) (PMMA) Microparticles Containing 13% Indocyanine Green (<2 Microns)

Obtain sample of PMMA—indocyanine green melt (see melt method protocol above); Add 20 ml of DCM to (1); vortex for 2 minutes; Pour 1 L of petroleum ether into a 1 L glass beaker; Add DCM to petroleum ether and allow suspension to sit for 5 minutes. Collect the particles using the same method illustrated in Example 1.

### Example 39

### Poly(methyl methacrylate) (PMMA) Microparticles Containing 13% Iron (III) Oxide (<2 Microns)

Obtain sample of PMMA—iron (III) oxide melt (see melt method protocol above); Add 20 ml of DCM to (1); vortex for 2 minutes; Pour 1 L of petroleum ether into a 1 L glass beaker; Add DCM to petroleum ether and allow suspension to sit for 5 minutes. Collect the particles using the same method illustrated in Example 1.

Melt—Solvent Evaporation (mSE) Method

### Example 40

### Polystyrene (PS) Microparticles Containing 13% Brilliant Blue G (<4 Microns)

Obtain sample of PS—brilliant blue G melt (see melt method protocol above); Add 15 ml of DCM to (1); vortex for 2 minutes; Pour 100 ml of surfactant, 0.2% PVA(MW˜25,000 Da, 88% hydrolyzed), into a 200 ml Virtis® flask; Place PVA under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; Add DCM-PVA to PVA; let mix for 24 hours.

Pour slurry of particles into 50 ml Eppendorf® tubes. Screw cap on and centrifuge for 20 minutes at 4,000 RPM; Aspirate off PVA solution using a 50 ml pipette tip; Flash freeze and lyophilize for 48 hours.

### Example 41

### Polystyrene (PS) Microparticles Containing 13% β-Carotene (<4 Microns)

Obtain sample of PS—β-carotene melt (see melt method protocol above); Add 15 ml of DCM to (1); vortex for 2 minutes; Pour 100 ml of surfactant, 0.2% PVA(MW˜25,000 Da, 88% hydrolyzed), into a 200 ml Virtis®) flask; Place PVA under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; add DCM to PVA; let mix for 24 hours;Collection of PS microparticles. Collect the particles using the same method illustrated in Example 1.

### Example 42

### Polystyrene (PS) Microparticles Containing 13% Copper (II) Phthalocyanine (<4 Microns)

Obtain sample of PS—copper(II) phthalocyanine melt (see melt method protocol above); Add 15 ml of DCM to (1); vortex for 2 minutes; Pour 100 ml of surfactant, 0.2% PVA(MW˜25,000 Da, 88% hydrolyzed), into a 200 ml Virtis® flask; Place PVA under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; Add DCM to PVA; let mix for 24 hours;Collection of PS microparticles. Collect the particles using the same method illustrated in Example 1.

### Example 43

### Polystyrene (PS) Microparticles Containing 13% Indocyanine Green (<4 Microns)

Obtain sample of PS—indocyanine green melt (see melt method protocol above); Add 15 ml of DCM to (1); vortex for 2 minutes; Pour 100 ml of surfactant, 0.2% PVA(MW˜25,000 Da, 88% hydrolyzed), into a 200 ml Virtis® flask; Place PVA under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; Add DCM to PVA; let mix for 24 hours;

Collect the particles using the same method illustrated in Example 1.

### Example 44

### Polystyrene (PS) Microparticles Containing 13% Iron (III) Oxide (<4 Microns)

Obtain sample of PS—iron (III) oxide melt (see melt method protocol above); Add 15 ml of DCM to (1); vortex for 2 minutes; Pour 100 ml of surfactant, 0.2% PVA(MW˜25,000 Da, 88% hydrolyzed), into a 200 ml Virtis® flask; Place PVA under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; Add DCM to PVA; let mix for 24 hours.

Collect the particles using the same method illustrated in Example 1.

### Example 45

### Poly(methyl methacrylate) (PMMA) Microparticles Containing 13% Brilliant Blue G (<3 Microns)

Obtain sample of PMMA—brilliant blue G melt (see melt method protocol above); Add 15 ml of DCM to (1); vortex for 2 minutes; Pour 100 ml of surfactant, 0.2% PVA(MW˜25,000 Da, 88% hydrolyzed), into a 200 ml Virtis® flask; Place PVA under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; Add DCM to PVA; let mix for 24 hours.

Collect the particles using the same method illustrated in Example 1. Pour slurry of particles into 50 ml Eppendorf® tubes. Screw cap on and centrifuge for 10 minutes at 1,000 RPM; Aspirate off supernatant and collect into a second 50 ml Eppendorf® tube. Screw cap on and centrifuge for 5 minutes at 4,000 RPM; Aspirate off supernatant; flash freeze and lyophilize pellet for 48 hours.

### Example 46

### Poly(methyl methacrylate) (PMMA) Microparticles Containing 13% β-Carotene (<4 Microns)

Obtain sample of PMMA—β-carotene melt (see melt method protocol above); Add 15 ml of DCM to (1); vortex for 2 minutes; Pour 100 ml of surfactant, 0.2% PVA(MW˜25,000 Da, 88% hydrolyzed), into a 200 ml Virtis® flask; Place PVA under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; Add DCM to PVA; let mix for 24 hours.

Collect the particles using the same method illustrated in Example 1.

### Example 47

### Poly(methyl methacrylate) (PMMA) Microparticles Containing 13% Copper(II) Phthalocyanine (<1 Micron)

Obtain sample of PMMA—copper(II) phthalocyanine melt (see melt method protocol above); Add 15 ml of DCM to PMMA; vortex for 2 minutes; Pour 260 ml of surfactant, 0. 1% PVA (MW˜25,000 Da, 88% hydrolyzed), into a 400 ml beaker; Place PVA under Virtis® Cyclone set to “10”; let mix for 60 seconds; Add DCM to PVA; let mix for 30 minutes.

Pour slurry of particles into 50 ml Eppendorf® tubes. Screw cap on and centrifuge for 10 minutes at 1,000 RPM; Aspirate off supernatant and collect into a second 50 ml Eppendor® tube. Screw cap on and centrifuge for 5 minutes at 4,000 RPM; Apspirate off supernatant; flash freeze and lyophilize pellet for 48 hours.

### Example 48

### Poly(methyl methacrylate) (PMMA) Microparticles Containing 13% FD&C Red #27 (<1 Micron)

Obtain sample of PMMA—FD&C red #27 melt (see melt method protocol above); Add 15 ml of DCM to (1); vortex for 2 minutes; Pour 260 ml of surfactant, 0.1% PVA (MW˜25,000 Da, 88% hydrolyzed), into a 400 ml beaker; Place PVA under Virtis® Cyclone set to “10”; let mix for 60 seconds; Add DCM to PVA; let mix for 30 minutes;

Collect the particles using the same method illustrated in Example 8.

### Example 49

### Poly(methyl methacrylate) (PMMA) Microparticles Containing 13% Indocyanine Green (<4 Microns)

Obtain sample of PMMA—indocyanine green melt (see melt method protocol above); Add 15 ml of DCM to (1); vortex for 2 minutes; Pour 100 ml of surfactant, 0.2% PVA(MW -25,000 Da, 88% hydrolyzed), into a 200 ml Virtis® flask; Place PVA under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; Add DCM to PVA; let mix for 24 hours. Collect the particles using the same method illustrated in Example 1.

### Example 50

### Poly(methyl methacrylate) (PMMA) Microparticles Containing 30% Iron (III) Oxide (<4 Microns)

Obtain sample of PMMA—iron (III) oxide melt (see melt method protocol above); Add 15 ml of DCM to (1); vortex for 2 minutes; Pour 100 ml of surfactant, 0.2% PVA(MW˜25,000 Da, 88% hydrolyzed), into a 200 ml Virtis® flask; Place (3) under Virtis® Cyclone set to “30”; let mix for 60 seconds; Add DCM to PVA; let mix for 15 minutes; Pour 160 ml of 0.2% PVA (MW≈25,000 Da; 88% hydrolyzed) into a 400 ml beaker. Place beaker under impeller (approximately 0.5 cm from bottom of beaker) and set impeller speed at 3,000 RPM; Add DCM to PVA; let mix for 24 hours. Collect the particles using the same method illustrated in Example 1.

### Example 51

### Poly(l-lactic acid) (PLA) Microparticles Containing 30% Carbon (<2 Micron)

Obtain sample of PLA—carbon melt (see melt method protocol above); Add 15 ml of DCM to (1); vortex for 2 minutes; Pour 260 ml of surfactant, 0.1% PVA (MW˜25,000 Da, 88% hydrolyzed), into a 400 ml beaker; Place PVA under Virtis® Cyclone set to “0”; let mix for 60 seconds; Add PVA to DCM; let mix for 4 hours.

Pour slurry of particles into 50 ml Eppendorf® tubes. Screw cap on and centrifuge for 5 minutes at 1,000 RPM; Aspirate off supernatant and collect into a second 50 ml Eppendorf® tube. Screw cap on and centrifuge for 5 minutes at 4,000 RPM; Aspirate off supernatant; flash freeze and lyophilize pellet for 48 hours.

### Example 52

### Poly(l-lactic acid) (PLA) Microparticles Containing 13% Copper(II) Phthalocyanine (<2 Micron)

Obtain sample of PLA—copper(II) phthalocyanine melt (see melt method protocol above); Add 15 ml of DCM to PVA; vortex for 2 minutes; Pour 260 ml of surfactant, 0.1% PVA (MW˜25,000 Da, 88% hydrolyzed), into a 400 ml beaker; Place PVA under Virtis® Cyclone set to “0”; let mix for 60 seconds. Add DCM to PVA; let mix for 4 hours. Collect the particles using the same method illustrated in Example 12.

### Example 53

### Poly(l-lactic acid) (PLA) Microparticles Containing 13% FD&C Black #2 (<2 Micron)

Obtain sample of PLA—FD&C black #2 melt (see melt method protocol above); Add 15 ml of DCM to (1); vortex for 2 minutes; Pour 260 ml of surfactant, 0.1% PVA (MW 25,000 Da, 88% hydrolyzed), into a 400 ml beaker; Place PVA under Virtis® Cyclone set to “0”; let mix for 60 seconds; Add DCM to PVA; let mix for 4 hours. Collect the particles using the same method illustrated in Example 12.

### Solvent Evaporation (SE) Method

### Example 54

### Poly(l-lactic Acid) (PLA) Microparticles Containing 30% Carbon (<2 Microns)

Weigh 500 mg of PLA (2,000 MW) in a 20-ml glass scintillation vial; Weigh 215 mg of carbon into a second 20-ml glass scintillation vial; Add 15 ml of DCM to (2); Pour DCM to PLA and vortex for 30 seconds; Pour 250 ml of surfactant, 1.0% PVA (MW≈25,000 Da; 88% hydrolyzed), into a 1-L Virtis® flask; Pour 100 ml of 0.5% PVA (MW≈25,000 Da; 88% hydrolyzed) into an 800 ml beaker. Place beaker under impeller (approximately 0.5 cm from bottom of beaker) and set impeller speed at 3,000 RPM. Set Virtis® Cyclone to “55” (13,750 RPM); let mix for 60 seconds, then add 100 microliters of 1-octanol;Allow emulsion to set for 5 minutes; Shake PVA well and add to Cyclone. Let mix for 15 minutes; Pour contents from Virtis® flask into (6) and let stir for approximately 24 hours.

Pour slurry of particles into 50 ml Eppendorf® tubes. Screw cap on and centrifuge for 20 minutes at 4,000 RPM (3345×g); Aspirate off PVA solution using a 50 ml pipette tip; Add 40 ml distilled water to tubes; mix and shake well until particles resuspend in distilled water (NOTE: sonication will likely be necessary to break up particle aggregates stuck to the bottom of the tubes); Screw cap back on and centrifuge for an additional 20 minutes at 4,500 RPM;

Aspirate off distilled water using a 50 ml pipette tip; Add 40 ml distilled water to tubes; mix and shake well until particles resuspend in distilled water; Screw cap back on and centrifuge for an additional 20 minutes at 4,000 RPM (3345×G); Aspirate off distilled water using a 50 ml pipette tip; combine slurry of particles into one or two tubes, flash freeze and lyophilize for 48-72 hours.

### Example 55

### Poly(l-lactic acid) (PLA) Microparticles Containing 30% FD&C Black #2 ( <2 Microns)

Weigh 500 mg of PLA (2,000 MW) in a 20-ml glass scintillation vial; Weigh 215 mg of FD&C black #2 into a second 20-ml glass scintillation vial; Add 15 ml of dye to DCM; Pour DCM into PLA and vortex for 30 seconds; Pour 250 ml of surfactant, 1.0% PVA (MW≈25,000 Da; 88% hydrolyzed), into a 1-L Virtis® flask; Pour 100 ml of 0.5% PVA (MW≈25,000 Da; 88% hydrolyzed) into an 800 ml beaker. Place beaker under impeller (approximately 0.5 cm from bottom of beaker) and set impeller speed at 3,000 RPM. Set Virtis® Cyclone to “55” (13,750 RPM); let mix for 60 seconds, then add 100 microliters of 1-octanol; Allow emulsion to set for 5 minutes; Shake PVA well and add to Cyclone. Let mix for 15 minutes; Pour contents from Virtis® flask into emulsion and let stir for ≈24 hours. Collect the particles using the same method illustrated in Example 1.

1. A method for enhancing uniformity of dispersion and increasing loading of nanoparticles in a polymeric microparticle comprising
(a) forming a homogeneous dispersion of nanoparticles in a polymer solution optionally containing surfactant,
(b) sonicating or exposing the polymer solution to shear forces effective to crease a stabilized dispersion of the polymer solution wherein the nanoparticles remain suspended for at least thirty minutes, preferably at least two hours, and
(c) forming microcapsules by a process selected from the group consisting of spray drying, solvent evaporation, interfacial polymerization, hot melt encapsulation, phase separation encapsulation, spontaneous emulsion, solvent evaporation microencapsulation, solvent removal microencapsulation, coacervation, low temperature microsphere formation, and phase inversion nanoencapsulation.

2. The method of claim 1 wherein the nanoparticle-polymer solution is mixed with an open blade or rotor stator type mixer at between 5000 and 25,000 RPM.

3. The method of claim 1 wherein the nanoparticle-polymer solution is milled with a concentric shaft type device.

4. The method of claim 1 wherein the polymer solvent is removed by solvent evaporation or extraction.

5. The method of claim 1 comprising
(a) dispersing nanoparticles to be encapsulated in a molten polymer,
(b) allowing the mixture of step (a) to cool to room temperature,
(c) dissolving the mixture of step (b) in an organic solvent,
(d) mixing the solution of step (c) using continual high shear stirring at between 3000-30,000 RPM and/or sonication for between about 1 minutes and about 30 minutes, and
(e) adding the result of step (d) to a non-solvent for the polymer, and optionally evaporating the solvent, to further precipitate the polymer in the form of microparticles containing a uniform distribution of nanoparticles.

6. The method of claim 5 wherein the non-solvent comprises surfactant in a concentration from about 0.001% w/v (surfactant to non-solvent) to about 5% w/v (surfactant to non-solvent).

7. The method of claim 1 comprising a surfactant, wherein the surfactant is selected to have a similar hydrophobicity or hydrophilicity as the nanoparticles to be incorporated.

8. The method of claim 7, wherein the non-solvent is water or a mixture of water and organic solvents which are miscible with water.

9. The method of claim 8, wherein the surfactant is selected from the group consisting of polyvinylalcohol, polyethylene glycol, and combinations thereof.

10. The method of claim 5, wherein the polymer is a water-insoluble polymer.

11. The method of claim 9, wherein the non-solvent is an oil comprising a surfactant.

12. The method of claim 11, wherein the surfactant is selected from the group consisting of fatty acids, cholesterol and cholesterol derivatives, polysorbates, lecithin and combinations thereof.

13. The method of claim 5, wherein the nanoparticles are in the polymer when it is melted.

14. The method of claim 1, wherein the microparticles are formed by precipitation.

15. The method of claim 1 wherein the polymer is selected from the group consisting of biodegradable polymer and non-biodegradable polymer.

16. The method of claim 1 comprising forming a dispersion having uniformly dispersed therein nanoparticles of less than one micron in diameter.

17. The method of claim 1 comprising forming microparticles having a diameter of less than 10 microns, between about 2 and about 3 microns, or between about 1 and about 2 microns.

18. The method of claim 1 wherein the nanoparticles comprise an agent selected from the group consisting of dye or chromophore, cosmetic, therapeutic, prophylactic, diagnostic, fragrant, nutraceutical, and flavoring agents.

19. The method of claim 18 wherein the nanoparticles comprise uniformly distributed active agent(s) at a low loading of about 1-2% by weight of the nanoparticles.

20. The method of claim 18 wherein the nanoparticles comprise a dye or chromophore selected from the group consisting of fluorescent, chemiluminescent, reflective, amorphous, crystalline, spherical or reflective particles, metals, magnetic, and colorless activatable particles.

21. The method of claim 1 wherein the microparticles comprise between approximately 1 and 80% nanoparticle by weight of microparticle, between approximately 10 and 60% nanoparticle by weight of microparticle, or between approximately 10 and 40% nanoparticle by weight of microparticle.

22. The method of claim 1 wherein the microparticles are made by solvent evaporation microencapsulation using a high oil to aqueous phase ratio effective to produce particles in combination with surfactant effective to improve dispersion of the pigment in the oil phase.

23. The method of claim 22, wherein the surfactant is soluble in the organic oil phase.

24. The method of claim 22 wherein the solvent is a mixed solvent containing a mixture of at least one water-immiscible solvent and water containing a surfactant in a ratio of from about 0.001% to about 5% surfactant (v/v), wherein the ratio of the water-immiscible solvents to the water is from 5 to 95%.

25. The method of claim 1, comprising a surfactant wherein the surfactant is selected from the group consisting of fatty acids, cholesterol and cholesterol derivatives, polysorbates, lecithin and combinations thereof.

26. Polymeric microparticles having nanoparticles uniformly dispersed therein, produced by the method of claim 1, the microparticles having a homogenous size distribution, uniform nanoparticle distribution and high nanoparticle loading.

27. The microparticles of claim 26 suitable for use in tissue marking, cosmetic use, therapeutic uses, or plastic surgery.

28. A method of marking skin comprising administering into or onto the skin the microparticles of claim 27.
